(19) United States
(12) Patent Application Publication
(10) Pub. No.: US 2025/0143604 A1
(43) Pub. Date: May 8, 2025

(54) BIOMETRIC SENSING AND MONITORING SYSTEMS AND DEVICES EMPLOYING A MICROWAVE SENSOR UNIT AND METHODS FOR USING THE SAME

(71) Applicants: Peter Edward Light, Sherwood Park (CA); Masoud Baghelani, Edmonton (CA)

(72) Inventors: Peter Edward Light, Sherwood Park (CA); Masoud Baghelani, Edmonton (CA)

(21) Appl. No.: 18/942,347

(22) Filed: Nov. 8, 2024

Related U.S. Application Data
(60) Provisional application No. 63/597,067, filed on Nov. 8, 2023.

(51) Int. Cl. A61B 5/145; A61B 5/00; A61B 5/0507

(52) U.S. Cl. CPC A61B 5/145; A61B 5/0004; A61B 5/0507; A61B 2562/0228

(57) ABSTRACT

Provided herein is a non-invasive biometric sensing and monitoring device that may be employed in a non-invasive method with the potential for comprehensive analysis and data processing. The non-invasive biometric sensing and monitoring device may be employed for determining a characteristic of a state of a wearer using the device based on a value of a biomolecule within a body tissue of the user. The device may include a housing for retaining one or more components of the biometric device. The components may may include a substrate, such as a printed circuit board, and may further include one or more of an signal generator, a microwave structure based sensor unit, a filter unit, a receiver component, an analog to digital converter, a control unit, one or more buffers, a communications module and controller as well as an analytics system.

### FIELD OF THE DISCLOSURE

The present disclosure relates generally to the detection, identification, and/or measurement of biomarkers. In particular, presented herein is a device, system, and method for implementing the same for monitoring one or more biomarkers using the microwave technologies disclosed herein. In various instances, the disclosed devices may be applied to a body part, such as an arm or wrist, of an individual in need of physiological monitoring, such as for the tracking a level of one or more body biomolecules, e.g., glucose. In such instances, once stably positioned on the body, the microwave based sensing and monitoring device may be employed in real-time, or near real-time, monitoring and tracking in a manner that allows allow for non-invasive, continuous or semi-continuous biomarker measurement and tracking.

In particular instances, an exemplary device of the system may include one or more microwave emitting sensors, such as a single or multiple microwave structure based sensor, which sensor is configured for transmitting one or more transmission signals, e.g., of high frequency, both through a conductive element of the device and into a body tissue. Likewise, the sensor may be configured for measuring the propagated energy through and around the conductive element of the device, especially with regard to measuring a portion of that energy that propagates through the dielectric media proximate the conductive element. Specifically, the device may be configured for receiving and measuring the energy transmitted through both the conductive element and the dielectric media proximate the conductive element so as to compare the results thereof and thereby determine one or more transmission and/or permittivity coefficients of the conductive element in relation to the associated dielectric medium. In various embodiments, the conductive element may be a microwave structure.

### BACKGROUND OF THE DISCLOSURE

In the field of medical diagnostics and healthcare, various detection and monitoring devices as well as the methods for using the same have been developed for biomarker measurement. In the majority, these include invasive and semi-invasive techniques, such as finger-pricking for blood glucose measurement and Continuous Glucose Monitoring (CGM) systems for diabetes management. While these methods have been instrumental in providing valuable information, they suffer from limitations such as discomfort, inconvenience, and high costs due to their invasive or semi-invasive nature. Furthermore, these methods are typically restricted to monitoring a single specific biomarker, lacking the capability for simultaneous or separate monitoring of multiple biomarkers.

One set of such medical diagnostic and healthcare detection and monitoring devices include microwave sensors. Such microsensor devices have been known and used for various applications. However, historically, such devices typically rely on resonance frequency analysis for the performance of measurements. Such devices are useful, but their usefulness is limited to the detection of variations observed in controlled environments. Consequently, in use, such resonance devices are limited to measurements of one factor, e.g., one variable parameter, to derive a sensed condition within a controlled environment.

For instance, such sensors could be used to determine a change of a concentration of a molecule within a stable aqueous environment, such as the change of a given ketone within water. Particularly, in a controlled aqueous environment, the change in concentration of a molecule could be detected. In this regard, such sensor devices include a resonator structure that can be placed in contact with a dielectric environment, and as a parameter within that environment changes, along a single dimension, e.g., concentration, the change in that environment, such as with respect to permittivity, can be detected by the sensor device, such as by detecting the change in resonance frequency.

Specifically, in this configuration, a change in the ambient environment due to an increase or decrease in concentration of a single molecule within that environment, can be detected by a resonance sensor that is attuned to that environment, based on the resonance frequency of the sensor structure and that of ambient material. In this regard, the correspondence between the sensor structure and the ambient environment define an effective permittivity, such that when the permittivity of the environment changes, a concomitant change in the effective permittivity of the structure also changes in a detectable manner. Therefore, such sensors are useful for determining that there has been a change in an environment, but they are not so useful as to be able to determine what has changed and why.

n view of these limitations, such sensors could not feasibly be used for sensing a plurality of biomolecules within the ever changing environmental milieu of the interstitial fluid within the tissues of the body. Hence, such sensors could be used to determine that a change of an amount of a single electrolyte within a solution has occurred, such as based on a resonance frequency shift. However, when the system moves from such a simple test configuration to a complex environment, e.g., with more than one variable, such sensors cannot easily be used.

### SUMMARY OF THE DISCLOSURE

Disclosed embodiments encompass the development of a novel, non-invasive, multi-feature electromagnetic radiation, e.g., microwave, transmission device and system, as well as methods of using the same, for real-time, or near real-time, biomarker detecting, sensing, tracking, and/or monitoring. In particular iterations, the electromagnetic radiation may be in the form of one or more high frequency transmission signals, including one or more of radio waves or microwaves, and the like. Particularly, the devices set forth herein are configured for emitting and transmitting a signal through and around a conductive element, such as into a dielectric medium proximate the conductive element, for instance, a liquid solution or into the skin of a living tissue, so as to produce a detectable response in that medium, e.g., tissue. In such instances, the conductive element may be a microwave structure placed in proximity to a body tissue, and upon activation, a structured electromagnetic radiation, e.g., microwave, mediated skin response may be observed. Likewise, the electromagnetic radiation emitting device may also be configured for receiving one or more of confined, reflected, as well as non-confined transmission signal back from the tissue so as to obtain and measure a broad dielectric spectrum that is reflective of the structured skin response.

Particularly, in various embodiments, the devices of the disclosure, e.g., microwave based sensing and monitoring devices, are configured for measuring structured responses of tissues of the body that are mediated by the transmission of electromagnetic, e.g., microwave, signals through and around a microwave structure. For these purposes, the sensing devices of the system include a sensing element that may be configured as one or more of an transmission signal emitter and/or receiver. In particular embodiments, the transmitter, e.g., input circuitry, and receiving, e.g., readout circuitry, elements may be composed of one or more frequency synthesizers and power detectors, which may be separated one from the other by a conductive element, e.g., a microwave structure, such as including one or more wire elements.

Specifically, in one aspect, provided herein is a non-invasive biometric sensing and monitoring device, such as for determining a characteristic of a state of a wearer using the device, for instance, based on a value of a biomolecule within a body tissue of the user. In various embodiments, the device may include a housing forming an encasement member. The encasement member may have a set of opposed surfaces offset from one another by a bounding member. For example, the housing may include a first and/or second set of encasement members, such as composed of opposed surfaces members, which may be separated from one another by the bounding member. In such instances, a first of the opposed surfaces may form a top surface and a second of the opposed surfaces may form a bottom surface. Together the plurality of opposed surfaces and the boundary member may bound a cavity, such as where the cavity is configured for retaining one or more components of the biometric sensing and monitoring device. The top and/or bottom encasement members may include or otherwise be associated with one or more insulation layers.

In this regard, the one or more components of the biometric sensing and monitoring device may include a substrate member, e.g., printed circuit board, which substrate member may also be associated with one or more insulation layers, such as those associated with a housing member. The components may further include one or more of an signal generator, a microwave structure based sensor unit, a filter unit, a receiver component, an analog to digital converter, a control unit, one or more buffers, as well as an analytics system, such as including one or more artificial intelligence modules, e.g., including a machine learning engine and/or an inference engine. In such instances, the substrate member may be an extended surface that includes a number of different layers, such as where the substrate member is positioned within the cavity and is configured for effectuating a coupling between one or more of the retained components.

As indicated, a signal generator, such as a frequency synthesizer unit, may be provided, such as where the signal generator is coupled to the substrate and is configured for generating a plurality signals. The generated signals may be of variable, high frequencies and may be communicated by the generator to the microwave structure in a manner to be transmitted across the microwave structure. Consequently, the sensing and monitoring device may include a sensor unit, such as a microwave based sensor component.

In this regard, a microwave structure unit may be provided and be positioned on the extended substrate layer within the cavity of the device housing. As indicated, the microwave structure unit may be coupled to the signal generator. For these purposes, the microwave structure unit may have a first port and a second port, such as where the first port is separated from the second port by an extended segment of a microwave structure. The microwave structure may be an extended microwave based conductive element that may have any suitable shape or design, and be composed of any suitable conductive material. For instance, the microwave structure may be straight, composed of one or multiple straight, e.g., linear, segments, whereby any of the linear segments can include or be coupled with one or more other linear segments by an angular segment, so as to have a convoluted or digitated configuration. Thus, the microwave structure may have a straight, linear, or curvilinear pathway between the first and second ports.

Each first and second port may include a positive and a negative lead or terminal that are separated from one another by a distance. As indicated, the first port may be configured to electronically couple the signal generator to the extended microwave structure and may be configured for receiving the first signal and for transmitting the first signal linearly forward along the pathway of the microwave structure from the first port to the second port. In various instances, the first port may further be configured for receiving a portion of the generated signal that becomes backwards propagated along the extended microwave structure, such as after having exited the first port and after having travelled along the microwave structure a second distance, then reverses course and is backwards propagated towards the first port.

Likewise, the second port may be configured for receiving a portion of the first impulse. For example, with respect to the transmission of the generated, e.g., interrogation, signal from the first port to the second, the energy of the generated and applied signal may be transmitted in a contained or non-contained manner. Specifically, a portion of the signal will be transmitted within the bounds of the microwave structure to be received directly at the second port. However, as this direct energy transmission occurs, a series of fringe fields will be produced, but will be propagated in a non-contained manner outside of the microwave structure. A portion of this non-contained signal will also be received at the second port. Both of these contained and non-contained signals can be received at port 2 of the microwave sensor unit so as to be measured by the receiver component.

Consequently, the biometric sensing, monitoring, and tracking device may include a receiver component, such as a power detector, that is coupled to the microwave structure and may be positioned on the extended substrate layer. The receiver component may be any device that is configured for receiving and/or measuring the various different portions of the transmitted signals. An analog to digital converter (ADC) may also be provided and be coupled to the receiver component. The ADC may be configured for converting an analog signal portion of the received transmission data, e.g., transmission response, into digital signal data, which can then be communicated by a suitably configured communications module to an analytics module of the system. A control unit for directing the operations of the other components of the system may be provided, such as for also receiving and conditioning the digital signal data into conditioned results data, such as prior to the communication of such results to an analytics module of the system, by an included communications unit of the system, for further evaluation thereby.

Accordingly, a unique aspect of the biometric sensing, monitoring, and tracking device is the microwave sensor unit that includes a novel microwave structure for implementing both confined and non-confined signal transmission. For instance, as indicated, the biomolecule sensing and monitoring device may include a microwave based sensor module, such as including a conductive element, e.g., microwave structure. In certain embodiments, the conductive element may be a microwave structure that, in certain implementations, may include one or more wires, whereby each wire based conducting element may be composed of the same or multiple different, independent, wires. In various embodiments, the wire based conductive element may be formed as a near-field antenna. In any instance, the conductive element may be an elongated structure that is coupled to both signal emission circuitry and signal readout circuitry on opposed sides of the structure

Specifically, the conductive element may be a microwave structure that includes one or more wire components. The conductive wire element may have any suitable configuration, shape, or design, for efficiently directing a transmission signal, e.g., microwaves, through the conductive element and into one or more body tissues proximate the conductive element. Unlike conventional devices that rely on sending and receiving microwave signals through two separate antenna structures, the present technology may employ a single conductive element, e.g., microwave structure, design to capture the broad dielectric spectrum of responses, thereby enhancing measurement accuracy and system simplicity.

For instance, the microwave structure based conductive element may be shaped so as to have a straight, curved, angular, and/or interdigitated configuration. The structure may be composed of one, two, three, or more wires, which one or more wires may be rounded, flat, or may be printed on a surface of a substrate. Since the sensing mechanism is completely non-invasive and detects variations through “physical sensing,” the choice of sensor material is highly flexible. It can be made from biosafe metals such as titanium, stainless steel, or gold, or from coated metals. The conductive element, with or without a coating layer, provides the necessary interface for the microwave signals to interact with the biological tissues or fluids, enabling accurate measurements.

This flexibility in design ensures that the sensor can be easily adapted for different applications and body locations. In such instances, the monitoring of the referenced coefficients may be performed using dedicated circuitry that is adapted for scanning frequencies from about 1 to about 10 to about 100 MHz or less to about 1 or about 5 or 10 GHz to about 20 or 30 or 50 GHz or more. For these purposes, the microwave based conductive structured element may be configured for efficiently directing transmitted signal through the conductive element, while at the same time as directing propagated microwave signals, e.g., fringe fields, into the body tissue.

As described herein, the excitation and readout circuitry of the sensing devices presented herein can measure the confined and non-confined transmission and/or reflection response of the sensor over a broad frequency spectrum, ranging from less than 100 MHz to over 10 GHz. By leveraging one or more broad dielectric spectrums obtained from measuring electromagnetic, e.g., microwave, radiation structured responses, the present technologies and systems offer enhanced sensitivity, selectivity, readout circuitry, and data processing capabilities. This non-invasive, biomolecule measuring technology overcomes many of the limitations of invasive or semi-invasive methods, offering the capability to monitor multiple biomarkers simultaneously or separately, providing comprehensive health insights without the discomfort or inconvenience associated with traditional diagnostic techniques.

Various of the system's distinguishing features include advanced data processing algorithms and a unique calibration methods that enhance measurement accuracy and reliability, establishing its prominence in the field of medical diagnostics. Beyond healthcare applications, the technology's adaptability proves valuable in the sports industry for precise monitoring of athletes' physiological markers, optimizing performance. Furthermore, its potential as a wearable device for the general public introduces opportunities for personalized health tracking, preventive care, and early detection of health issues.

A described herein, another unique and innovative advancement of the system lies in its non-invasive nature and the comprehensive data collection and analytics it provides. Traditional biomarker monitoring techniques often require invasive procedures, such as blood tests, which can be uncomfortable and inconvenient for patients. By contrast, the present microwave-based system offers a painless and convenient alternative, with the ability to provide continuous or semi-continuous monitoring. Accordingly, in another aspect, provided herein is a microwave based biomolecule sensing and monitoring system.

For example, in particular iterations, the system's hardware, including the excitation circuitry, microwave sensing element, readout circuitry, and processing unit, is configured for being consistent across the various different applications to which the disclosed devices may be employed. This uniformity simplifies the design and manufacturing process, making the system more cost-effective and easier to deploy. Additionally, such simplicity allows for the simultaneous or separate monitoring of multiple biomarkers, depending on the specific needs of the user. Particularly, in various embodiments, the system's hardware configuration may be variable, but may for example, include a control unit. The control unit can include a processor coupled to a memory and one or more of: (i) communication interface, and (ii) an input interface.

One or more processors may be included having one or more electronic devices that is/are capable of reading and executing instructions stored on a memory to perform operations on data, which may be stored on a memory or provided in a data signal. The term “processor” includes a plurality of physically discrete, operatively connected devices despite use of the term in the singular. Non-limiting examples of processors include devices referred to as microprocessors, microcontrollers, central processing units (CPU), and digital signal processors.

Memory can be included wherein the memory includes a non-transitory tangible computer-readable medium for storing information in a format readable by a processor, and/or instructions readable by a processor to implement an algorithm. The term “memory” includes a plurality of physically discrete, operatively connected devices despite use of the term in the singular. Non-limiting types of memory include solid-state, optical, and magnetic computer readable media. Memory may be non-volatile or volatile. Instructions stored by a memory may be based on a plurality of programming languages known in the art, with non-limiting examples including the C, C++, Python™, MATLAB™, and Java™ programming languages.

A communication module having a communication interface may be provided, whereby the communication interface includes a cellular modem and antenna for wireless transmission of data to a communications network. It may also utilize other communication standards such as Wi-Fi, Bluetooth, etc. An input interface having any known interface for receiving user inputs (e.g., buttons), can be provided.

To that end, it will be understood by those of skill in the art that references herein to user device as carrying out a function or acting in a particular way imply that processor is executing instructions (e.g., a software program) stored in memory and possibly transmitting or receiving inputs and outputs via one or more interfaces. In some examples, memory can store the learning software.

With regard to the overall structure of the data processing system, the system is similar for both training and calibrating the AI model and the calculation of the biomarker of interest. The data from the microwave sensor is acquired using the readout circuitry. This data contains the frequency points over the defined frequency span and is provided at certain time intervals (e.g., every 4 seconds or every 10 seconds). The data is fed to the preprocessing and data trimming engines, responsible for data cleaning, detection of movement, detection of interferences, etc.

The trimmed and cleaned data is then fed to the feature selection algorithm, where the proper features for each biomarker are selected. This section mainly works based on the data considered for training the model. The best feature set is extracted to provide the best validation results. A custom-built genetic algorithm is involved in the feature selection process. The features could be the microwave sensor output at some distinct frequency points or mathematical combinations of a few of them.

The selected features are then applied to the calibrated/trained model to generate a value for the concentration of the biomarker of interest (e.g., glucose, lactate, etc.). The model is designed to detect variations in the biomarker of interest. This approach is believed to be acceptable since capturing the variation of the dielectric permittivity spectrum of the material under test (e.g, the human body) is more feasible non-invasively, as there are many person-to-person variations that could significantly impact the precision of the microwave sensing technology. Moreover, by extracting the variation trend (e.g., detection and calculation of the biomarker variation from one sampling time to the next), it is possible to calculate the exact concentration of the biomarker of interest with one initial value.

Accordingly, in various embodiments, the system may be configured for receiving one or more of the raw, conditioned, and/or preliminarily processed measurement data, and for analyzing the same. For these purposes, the system may include one or more analytics sub-systems for analyzing the received data so as to produce one or more biological condition results data. For example, the analytics system may include, one or more computing resources, such as a one or more remote, cloud-based server systems.

The server system itself may be composed of one or more processing units, such as including a plurality of central processing units (CPUs), graphics processing units (GPU's), and/or quantum processing units (QPUs). Additionally, the analytics system may include or otherwise access one or more artificial intelligence (AI) systems, which AI system may include one or more of machine learning and/or inference engine modules, such as for analyzing the microwave structured sensor measurement data and generating one or more predictions, e.g., calls, as to what a condition of the body of a wearer of the sensing and monitoring device is, such as based on an evaluation of the measured data.

Previously trained AI, including machine learning and/or inference algorithms typically attempt to extract concentration values of biomarkers of interest, but such calculations are too complex even for AI to perform. The present methods are different. As the concentration of a biomarker changes, it alters the dielectric properties of the tissue or fluid being monitored. These changes are detected as variations in the transmission responses of the sensor. The data collected is then processed by sophisticated AI and machine learning algorithms. Each biomarker is associated with a specific AI algorithm that has been trained to recognize patterns in the microwave responses that correlate with the biomarker's concentration levels.

The use of AI, including machine learning and inference generation in this system is a significant advancement over such inoperable attempts of the past. These algorithms can analyze complex datasets and identify subtle patterns that may be missed by traditional data processing methods. This capability enhances the accuracy and reliability of the biomarker measurements, providing more precise and actionable health information. A separate AI algorithm will be used for each biomarker, but the hardware, including the sensing element, readout circuitry, and processing unit, will remain the same.

In view of the foregoing, unlike traditional biomarker monitoring devices, systems and their methods of use that generate measurements based on specific resonances, the herein disclosed embodiments cannot only measure and monitor a number of specific resonances, the devices herein disclosed may be configured for considering a multiplicity of variations of the microwave generated structured tissue response over a broad frequency spectrum. This broad frequency spectrum allows the system to capture detailed information about the dielectric properties of the biological tissues or fluids, which are influenced by the concentration of biomarkers. In this manner, the performance of the device and the accuracy of its measurements may be enhanced.

As disclosed herein, unique data processing algorithms and novel AI determination and calibration methods are employed to enable the simultaneous or separate monitoring of multiple biomarkers using single or multiple microwave sensors. In this approach, the human body and its variations become an integral part of the sensor, as this method measures the sensor's characteristics (transmission and reflection responses). This is fundamentally different from approaches that consider the human body as a communication channel.

Consequently, presented herein, and according to at least one broad aspect, there is disclosed a non-invasive microwave-based biomarker monitoring system and method. The system comprises a microwave sensor or an array of sensors, configured to capture a broad dielectric spectrum of responses from biological tissues or fluids, enabling real-time or near real-time monitoring of biomarkers through the analysis of variations in the microwave structure response across a wide frequency range.

As disclosed above, the sensing element is placed on the skin, and as the concentration of a biomarker of interest (such as glucose, lactate, hydration, ketone, alcohol, etc.) changes, the overall reflection and transmission response of the sensor over the measured broadband changes in a detectable manner. Specifically, these changes can be detected by the above referenced microwave based sensor units. Hence, provided herein is a non-invasive biometric sensing and monitoring device that may be employed in a non-invasive method with the potential for comprehensive analysis and data processing, it is believed that the disclosed technology presents an advance in biomarker monitoring, enabling timely interventions, personalized healthcare, and improved patient outcomes.

The potential applications of this technology extend beyond traditional healthcare settings. For example, such devices and their methods of use have far ranging implications not only for the health monitoring and maintenance industries, but also represent a valuable asset for the sports industry, and provide a useful wearable device for the general public. In the sports industry, for example, the system can be used to monitor athletes' physiological markers, such as hydration levels and lactate concentrations, in real time. This information can be invaluable for optimizing performance and preventing injuries. Similarly, as a wearable device for the general public, the system can provide continuous health monitoring, helping individuals track their wellness and detect potential health issues early.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 illustrates an exemplary configuration for the non-invasive biometric sensing and monitoring device of the system, including a unique microwave sensor unit, in accordance with the teachings of the disclosure.

FIG. 2 illustrates an exemplary microwave sensing structure in accordance with the teachings of the disclosure.

FIG. 3 illustrates a human body portion, e.g., arm, wearing the non-invasive biometric sensing and monitoring device of FIG. 1.

FIG. 4 illustrates an exemplary system configuration employing the non-invasive biometric sensing and monitoring device of FIG. 1, in accordance with the methods taught in the disclosure.

FIG. 5A sets forth a block diagram exemplifying a methodology for using the non-invasive biometric sensing and monitoring device of FIG. 1 to generate digital measurement data.

FIG. 5B sets forth a block diagram exemplifying a methodology for analyzing, by the system set forth in FIG. 4, the digital measurement data derived by the methodology of FIG. 5A.

FIG. 5C sets forth a block diagram exemplifying an artificial intelligence training methodology to be employed by the system set forth in FIG. 4, in accordance with the teachings of the disclosure.

FIG. 6 sets forth a block diagram exemplifying a feature selection methodology in accordance with the teachings of the disclosure.

FIG. 7A sets forth a transmission response of the microwave sensor of FIG. 1, over a broad frequency spectrum.

FIG. 7B sets forth an exemplary plot for results of a human trial lactate measurement using calibrated non-invasive innovative biosensor, along with a comparison to fingerpick data.

FIG. 7C sets forth a digitized version of the transmission response of FIG. 7A, highlighting variations and interferences as small peaks in the response.

FIG. 7D sets forth an example plot of the microwave data measured by the microwave structure in FIGS. 1 and 2.

FIG. 7E presents microwave data measured by the microwave structure in FIGS. 1 and 2 comparing human trial glucose measurements using the non-invasive biosensor with finger-prick data, validating the sensor's accuracy and reliability.

FIG. 8 shows the Clarke Error Grid Analysis for glucose monitoring, with 100% of data within 20% of the reference standard sensor, extracted from FIG. 7E human trials.

### DETAILED DESCRIPTION

Traditional methods for biomarker analysis, such as blood sampling, can be invasive and uncomfortable for patients and may not provide real-time information. The development of non-invasive biomarker monitoring techniques has therefore long been a goal in the medical and healthcare industry. The need for accurate, non-invasive, and continuous biomarker measurement in real-time has led to extensive research and development efforts in the field.

As used herein, the term “microwave” means electromagnetic radiation fields with frequencies in the range between 1 MHz and 100 THz, corresponding to wavelengths between 300 m and 0.3 mm. However, the frequency span used in our invention is from less than 100 MHz to over 10 GHz. A “microwave structure” is an antenna, resonator, filter, or any other planar or non-planar design capable of transmitting and/or receiving, or fringing microwave radiation fields into and/or from a medium, such as a human body.

Microwave sensing technology offers many potential advantages and characteristics. However, typical microwave sensing technologies are subject to many limiting challenges, especially when applied for biomarker monitoring. For instance, one of the primary challenges faced by traditional microwave sensing technologies is their lack of selectivity for the biomarker to be measured. Traditional microwave sensing methods historically rely on specific resonances at selected frequencies within the microwave spectrum, limiting their ability to differentiate between different biomarkers accurately. This limitation compromises their selectivity, potentially leading to inaccuracies in measured biomarkers.

Moreover, precise detection of resonance frequencies is challenging. This challenge becomes more critical in applications where the dielectric properties of the target medium have very small changes, such as in biomarker measurement in animal or human tissue. This challenge arises from the small quality factor of microwave structures, which is usually tackled by utilizing energy-hungry solutions such as embedding the microwave resonators in the tank circuit of electronic oscillators.

Likewise, the complexity of signal processing also presents another challenge for monitoring biomarkers using traditional techniques. In particular, traditional microwave sensing technologies often struggle to provide sufficient information for meaningful calculations of biomarker levels. Issues such as noise, interference, movement artifacts, and background variations can adversely affect the accuracy and reliability of measurements. In view of the foregoing, to address these challenges and advance biomarker measurement, the present disclosure provides a novel non-invasive, multi-feature microwave structure. This innovation aims to provide accurate, non-invasive, and real-time monitoring capabilities, addressing the evolving needs of the medical and healthcare industry. In view of the forging, the presently disclosed embodiments address these and other fundamental challenges relating to traditional selectivity in biomarker analysis using microwave sensing technology within complex mediums, e.g., the human body. However, Rather than focusing solely on resonance frequencies, quality factors, or amplitude changes, the disclosed approach involves the measurement of amplitude and phase variations across a spectrum of frequencies, preferably a relatively broad spectrum.

As detailed herein and below, the disclosed devices, systems, and their methods of use involve the application and detection of microwave signals and their fringing microwaves, which produces a broad-spectrum frequency response the amplitude and phase response of which can be precisely measured by microwave structure. These detectable responses may include confined and non-confined transmission and/or reflection responses of the microwave structure. Embodiments herein rely on the unique dielectric spectrum of different biomarkers over a range of microwave frequencies. Each biomarker has a distinct dielectric signature, which may be detected and identified. For example, the broad-spectrum response of the biomarker may be used as the biomarker's signature, e.g., finger print, rather than relying on specific resonances at a selected frequency, which has been determined to be largely unworkable because of the inherent complexity thereof. This broad-spectrum approach allows for the measurement of numerous features in the reflection or transmission response of the microwave structure, which may be positioned in contact with the skin.

In its basic usage, provided herein are methods that involve the generation of data, e.g., measurements, by the unique sensing and monitoring devices of the system, which may be used for the collection and analysis of a large amount of measurement data. Specifically, these methods may include the collection of a multiplicity of data points, e.g., numbering in the hundreds or thousands, which offer unique insights into the response of the disclosed microwave sensing device. The inherent uniqueness of this microwave structure response, at every data point, effectively creates a distinctive feature vector for each specific biomarker, which uniqueness arises from the complex dielectric characteristics of biomarkers, their combinations, and the dielectric media that is proximate the sensing device that results in a multifaceted response pattern.

In at least one example, to further enhance accuracy and reliability, the system disclosed herein further provides a unique analytics sub-system that includes AI-based data processing algorithms. These algorithms are configured to account for noise, interference, and background variations so that biomarker measurements are more precise and reliable. This combination of the broad-spectrum multi-feature approach and advanced data processing capabilities enables real-time, or near real-time, continuous or semi-continuous, and non-invasive measurement of multiple biomarkers.

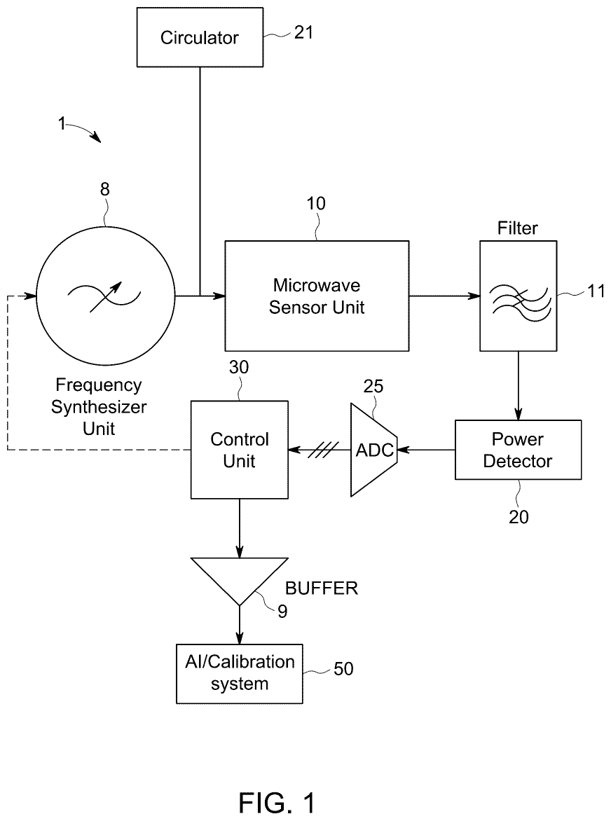

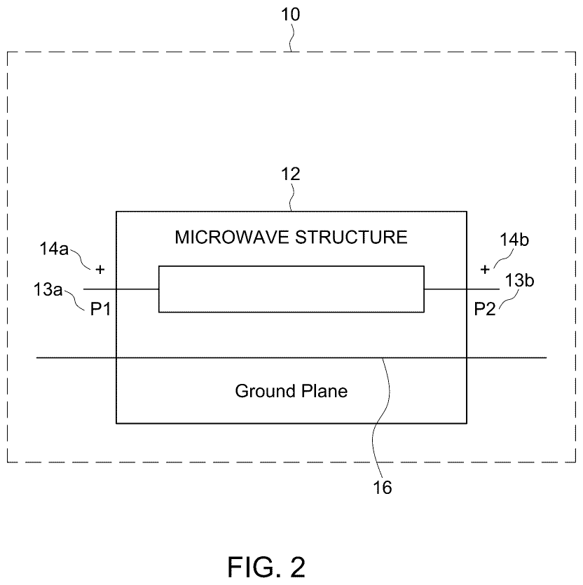

Accordingly, as can be seen with reference to FIG. 1, a non-invasive biometric sensing and monitoring device 1 is provided for use in determining one or more characteristics of a state of a living body, such as based on a value of a biomolecule within a tissue, such as skin, of that body. Specifically, as can be seen with respect to FIG. 2, the device 1 includes a microwave sensor unit 10 that contains a unique microwave structure 12, which extends between a first port 13a and a second port 13b.

Particularly, the first port 13a includes a positive pole or lead 14a and may include a negative lead. Likewise, the second port 13b also includes a positive terminal or pole 14b and may include a negative pole. As depicted, a ground plane 16 can be provided, such as in substitution for either one or both of the negative poles, for instance, where the ground plane 16 extends from the first port 13a and second port 13b. The positive terminals 13a,b are separated from the negative poles, e.g., the ground plane 16, by a distance, so as to create a potential difference, e.g., electrical potential difference, therebetween.

The device 1 also includes both excitation and readout circuitry. Such excitation circuitry may be embodied by any form of signal, e.g., power, generator 8, that is capable of generating and transmitting an electrical, e.g., voltage, electromagnetic, signal such as through a conductive element, such as wire. As depicted in FIG. 1, the excitation circuitry, e.g., signal generator, is embodied by a frequency synthesizer unit 8, and the conductive element is embodied in the microwave structure 12 of FIG. 2. The readout circuitry may be embodied by any form of signal detector unit 20 that is capable of detecting, measuring, quantifying, and/or characterizing the signal, such as with respect to its electrical and/or electromagnetic properties. As depicted in FIG. 1, the readout circuitry may be embodied by a power detector 20. The excitation circuitry, e.g., frequency synthesizer 8, is coupled to one end of the microwave structure 12, which microwave structure 12 is further coupled at its other end to the readout circuitry, e.g., a power detector unit 20.

Accordingly, in FIG. 1 a signal generator unit 8, such as a frequency synthesizer, is provided for generating one or more, e.g., a range, of frequencies that may be generated and applied to the microwave sensor unit 10, such as to the positive terminal 14a of the microwave structure 12. In particular implementations, the frequency synthesizer 8 may be a radio or microwave synthesizer that include an oscillator for producing one or more sets of stable frequencies from a single reference frequency in such a manner phase noise is minimized. In various embodiments, the synthesizer unit 8 may include one or more of a master oscillator, a voltage generator, counter, e.g., fixed counter, phase comparator, one or more filters, such as a low pass filter, a voltage control oscillator, a counter, and a micro controller or other frequency selection logic.

Hence, the frequency synthesizer may be employed for frequency generation, multiplication, division, mixing, direct digital synthesis, and the like. In this instance, the frequency synthesizer 8 may be configured for generating and supplying one or more electromagnetic waves to the microwave sensor unit 10. Particularly, when a voltage is applied to the frequency synthesizer the oscillator converts the direct current into an alternating current that, in turn, produces a periodic electromagnetic signal, such as in the form of a sinusoidal wave. The wavelength, frequency, and amplitude can all be finely controlled and modulated.

In this manner, a voltage is applied to the frequency synthesizer, which then produces an electromagnetic signal having a determined frequency. This signal is then conveyed to the positive input terminal of the first pole that is coupled to the microwave structure. The signal then gets transmitted along the microwave structure from the first terminal to the output terminal of the second port. At this point, the generated signal that is conveyed to the first port of the microwave structure is referenced herein as an interrogation signal, and it is basically a sinusoidal wave that has a determined frequency.

Accordingly, the frequency synthesizer generates a high frequency sinusoidal, electrical signal that gets applied to the first port and gets propagated from the first port to the second port along the microwave structure. This forward propagation of the applied signal is termed transmission. For example, in instances where the sensor unit 10 includes a two-port structure, transmission refers to how the input signal at the first, transmission, port 14a is transmitted to the output at the second, reception port 14b. This may be characterized by an S-parameter, e.g., S21, which represents the forward transmission coefficient. S21 is a complex ratio of the output voltage at Port 2 to the input voltage at Port 1, when Port 1 is excited and Port 2 is terminated with a matched load. It indicates how much of the signal is transmitted through the device.

FIG. 2 provides a two port sensor structure 10 that includes a first, e.g., transmission, port 14a that is separated by a microwave structure 12 from a second, e.g., reception, port 14b. Both ports share a ground plane 16, but each could include separate individual ground terminals (not shown). The ground plane 16, shared between the transmission 14a and reception 14b ports, port 1 and port 2, can be considered as the reference node when measuring voltage. After the transmission signal traverses the microwave structure and reaches the second port 14b it may be conveyed to a power detector unit 20 where the various characteristics of the transmission signal can be measured and quantified. Once measured, quantified, and/or characterized, such as by the power detector 20, the transmission signal is then referred to as a transmission response.

The power detector 20 measures the difference between the input voltage at the transmission port 14a and the output voltage at the reception port 14b. The voltage is not the same between the two ports because as the electric signal travels from the first port 14a to the second port 14b, some of the signal is lost via the conduction process, e.g., conductive loss. Some of the signal makes it part way to the second port 14b but then reverses course and gets backwards propagated so as to return back to port 1 14b. When this happens, this is called the reflected signal, and it may be measured by third circuitry embodied as a circulator unit. Further, where the microwave sensor unit 1 is applied to a dielectric medium, such as a body tissue 100, some of the signal can be lost within that medium, which loss is termed dielectric loss.

For the applied interrogation signal that makes it from port 1 to port 2, there are two parts to this signal. The first part of the transmission signal stays confined within the microwave structure itself, and this is termed the confined signal, and it traverses through the microwave structure from port 1 to port 2, where it can be directly measured as part of the transmission response. However, some of this transmission signal gets propagated outside of the conductive structure, and so are not confined therein, but, nevertheless a portion of this non-confined signal makes it to port 2, 14b, and is considered part of the transmission response. These non-confined parts of the transmission signal are called fringe fields, and they circle around the microwave structure.

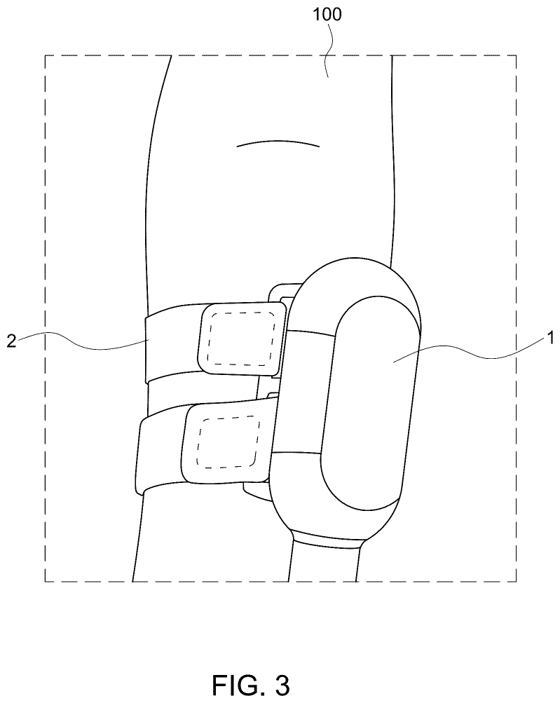

Consequently, when the biometric sensor unit 1 is placed next to a dielectric medium, such as a human body 100, as depicted in FIG. 3, the non-confined portion of the transmission signal propagates as one or more fringe fields through the body from port 1, 14a to port 2, 14b. These fringe fields travel laterally along the microwave structure, but they are circular. They leave the microwave structure, enter the body, and then return to microwave structure in a circular fashion. Therefore, this non-confined, fringe field transmission is not at the same speed as for the confined transmission signal, because these fringe fields have to be propagated through the body 100, whereby the body and its constituents can affect how much of the fringe field gets propagated and at what speed.

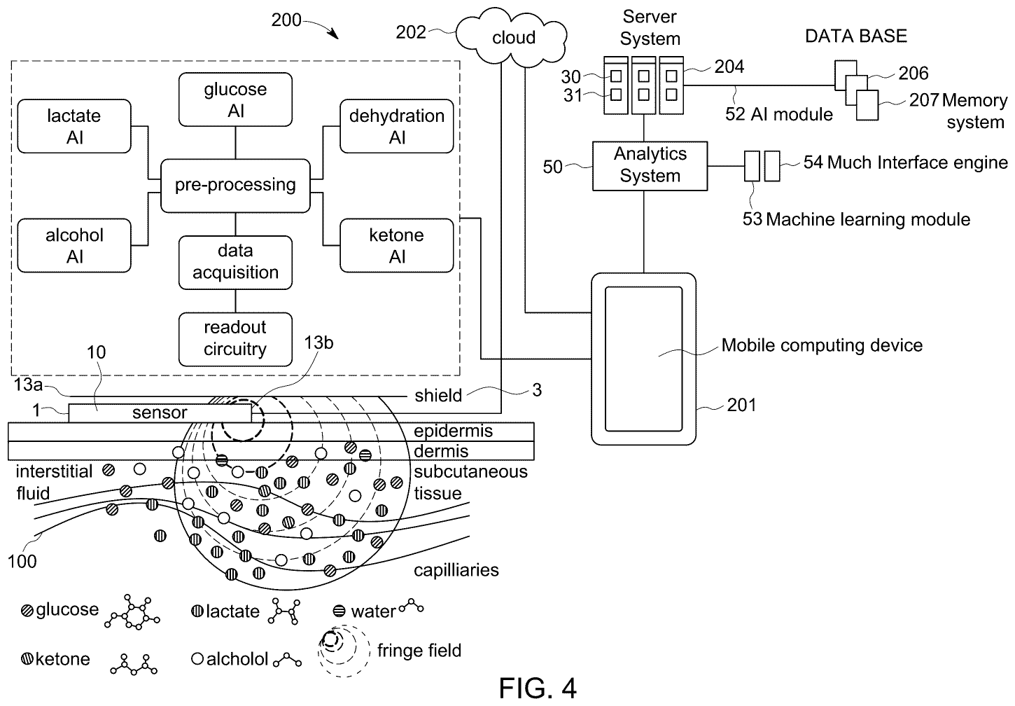

Specifically, the transmission of the propagated fringe fields is affected by the dielectric properties of the body to which the biometric sensing device is attached. For instance, when the biometric sensor unit 1, is attached to an arm of a body 100, via one or more attachment mechanisms, such as one or more straps, belts, chords, sleeves, patches, and the like, as depicted in FIG. 3, the fringing fields, as depicted in FIG. 4, will have to traverse through the skin from port 1, 13a, to port 2, 13b, but in doing so will be affected by all the properties of the skin and its constituents, e.g., cells, interstitial fluids, vessels, capillaries, biomolecules, which all have different dielectric characteristics.

Further, it has been determined by the inventors hereof that the characteristics of this fringe field transmission can be used to determine the presence, identity, concentration, and other characteristics of the biomolecules being present within the skin, e.g., based on their dielectric characteristics, the permittivity of the skin, how these biomolecules affect the skin, and by what frequency the transmission signal is being propagated. Exemplary biomolecules that can be detected and characterized in this manner include glucose, alcohol, lactate, ketones, and the like.

As shown with respect to FIG. 4, this raw transmission response data, measured by the power detector 20 coupled to port 2, 14b, of the microwave sensor unit 10, can be transmitted by a communications module 23 of the sensing device 1 to a remote processing center, such as including a server system 204 for analyzing and processing the raw transmission measurement data, as discussed herein below. Such remote server systems 204 may be cloud 202 accessible, whereby the server system 204 includes a communications module that is capable of receiving and processing the transmission data. In such instances, the server system 204 may include a number of control units 30 accessing a plurality of processing units 31, such as including one or more central processing units (CPUs), graphics processing units (GPUs), quantum processing units (QPUs), artificial intelligence processing units, and the like. These processing units 31 may be configured for preprocessing the received raw data, may analyzed the same, and may return results, e.g., preliminary results, of the analysis.

However, in some instances, the analytics system 50 may include, or may otherwise access, one or more artificial intelligence modules 52, which as described herein, may be configured for further processing the readout data so as to isolate and determine information about one or more of the aforementioned biomolecules and/or determining their effects on the body. Further, as explained herein, the AI module 52 may be configured for implementing one or more models, and thus, may include a machine learning engine 53 for generating and/or building the model, and may further include an inference engine 54 for implementing the model, such as for the purpose of characterizing one or more biomolecules or other constituents within the body tissues.

The system 200 may further include one or more databases 206, such as a cloud accessible database, whereby the system generated data may be stored and accessed. In such instances, one or more databases 206, which may be local or remote, having one or more memory systems 207, may be implemented for saving raw, pre-processed, processed, and relevant results data. Further, one or more of this data, such as biomolecule results data, may be presented, via a suitably configured client application running on a client computing device 201, for viewing by a user of the system, so as to identify, determine, monitor, and track various different biomolecules of the body and/or how those biomolecules are effecting the body. In such embodiments, the client computing device may be a desktop or mobile computing device, such as a mobile smart phone or tablet computing device 201.

Particularly, the presence and concentration of these biomolecules within the skin affect both the skin, such as with respect to its dielectric properties, but they may also affect the transmission properties of the fringing fields, such as with respect to their permittivity through the skin. Specifically, as the fringing fields are propagated through the skin, such as at high frequency, e.g., microwave frequencies, some of their energy gets absorbed by the skin and it constituent parts. More particularly, these constituent parts, especially the referenced biomolecules, may absorb and store some of this energy, such as within their chemical bonds, if that energy is of the right particular frequency.

For instance, in various embodiments, these high frequency transmission may range from about 100 MHz to about 10 GHz, such as from about 2−, 3−, 4−, 500 MHz to about 6, 7, 8 or even 9 GHz, including about 1 or 2 to about 4 or 5, including about 3 GHz. Specifically, as explained herein, because the dielectric permittivity experiences variation at the microwave range (within a microwave transmission regime), working at this frequency band provides unique features that enable the monitoring biomarkers, noninvasively. Consequently, the presence of these biomolecules within the tissues changes the dielectric permittivity of the tissues, e.g., skin, which affects the characteristics of the fringe fields as they propagate through the skin, which in turn changes the energy delivered at port 2, 14b, due to this fringe field transmission.

More specifically, permittivity is a measure of how much an insulating material, such as the tissues of a human body 100, opposes the formation of an electric field. The permittivity of a material is represented by epsilon, E, while the permittivity of a vacuum is represented ε₀, The ratio of these two values, E/ε₀, is known as the dielectric constant and is represented by kappa, K. Therefore, with regard to the dielectric characteristics of the body and its components, or any material for that matter, there are basically two different parts of dielectric permittivity.

First, there is the relative part of dielectric permittivity, and then there is the imaginary part of permittivity. It is the relative part of dielectric permittivity, e.g., epsilon R (εᵣ), which affects and modulates the capacitance of a material, including biomolecules. For instance, relative permittivity reflects a material's ability to store energy when an electric potential is applied across it. The relative permittivity describes the ease by which a dielectric medium, e.g., a biomolecule, may be polarized, and with regard to capacitance, the capacitance of a capacitor is proportional to εᵣ. In view of this, if ε₀ represents the permittivity of free space and ε represents the permittivity of a particular substance, such as a biomolecule, relative permittivity is symbolized as εᵣ or ε′, and for the imaginary part it is symbolized as epsilon prime, ε″. Relative permittivity is also called the dielectric constant. It is expressed as the following: εᵣ=ε/ε₀.

Consequently, for biomolecules, real permittivity is the building up of capacitance within the chemical bonds between the various elements and molecules of a biomolecule in accordance with their unique dielectric constant. In other words, real permittivity for a biomolecule is the storing of energy in the dipoles of the molecules, and in this regard the elements and molecules of the various biomolecules absorb the energy of the fringe fields as those fields pass through the tissues of the body, and thus, in this sense, they act as a biochemical capacitor. Hence, as the energy of the fringe field contacts the various tissues, skin, vessels, interstitial fluids, and biomolecules therein, these materials will oppose the propagation of the fringe field at very high, but different frequencies.

In essence, the polar molecules of these materials will rotate, e.g., around their chemical bonds, from one configuration, having less energy, to a second configuration, having more energy, thereby, in this manner acting as an energy store, e.g., a capacitor. Thus, all of the above referenced equations can be used to determine and characterize the various different biomolecules within the tissues of the body, especially with respect to their permittivity and effect on the referenced fringe fields. After interaction within a fringe field, as the molecule reconfigures itself so as to return to stability, this stored energy will be re-released, and will be free to be propagated towards the second port, 13b, after having made this slight detour.

Specifically, at the right frequency, based on the nature of the elements involved, these rotations will happen at a corresponding frequency to the applied frequency, and it is at this frequency the presence of the various biomolecules within the tissues can be detected, identified, and characterized, such as with regard to its concentration. For instance, if an interrogation signal is applied at 2.4 GHz, when the signal hits water, e.g., the interstitial fluids, within the tissues, such as during the positive phase of the pulse, energy will be absorbed by the water molecules, for example, and that energy will be released during the negative phase. Thus, in this manner, when various biomolecules within the tissue are contacted with a fringe field of the appropriate frequency, based on the dielectric constant of that biomolecule, the biomolecule will oscillate back and forth in correspondence with the received frequency of the electromagnetic field, which in this example would be at 2,400,000,000 times a second. Most of the stored energy will be returned to the environment and will be free to continue its propagation towards port 2, 13b, but a small portion of the energy will be lost due to friction caused by the rotations, e.g., dielectric loss. And it is this dielectric permittivity, leading to molecule reconfiguration, and dielectric loss that is unique to each biomolecule and can be used, as disclosed herein, not only to determine the presence of various biomolecules within the skin, but can also be used to qualify and quantify those molecules, such as with respect to determining their concentration.

In view of the forgoing, for the confined signal, substantially all of this energy will be transmitted to receiver component, e.g., power detector 20, at port 2, 13b. However, for the non-confined energy, e.g., the fringe fields, only a portion of this energy will be received at port 2, based on the dielectric permittivities of the tissue components with which the fringe fields have come into contact. Therefore, the capacitance at port 2 will change in a measurable way due to the interaction of the fringe fields with these biomolecules, and other tissue components, and this results in a variation in the amplitude of the non-confined fringe filed signal versus the confined transmission signal. Consequently, as different molecules absorb energy at different frequencies, these changes in signal characteristics, e.g., their waveform properties, can be used to detect and characterize the presence and concentration of these biomolecules within the tissues.

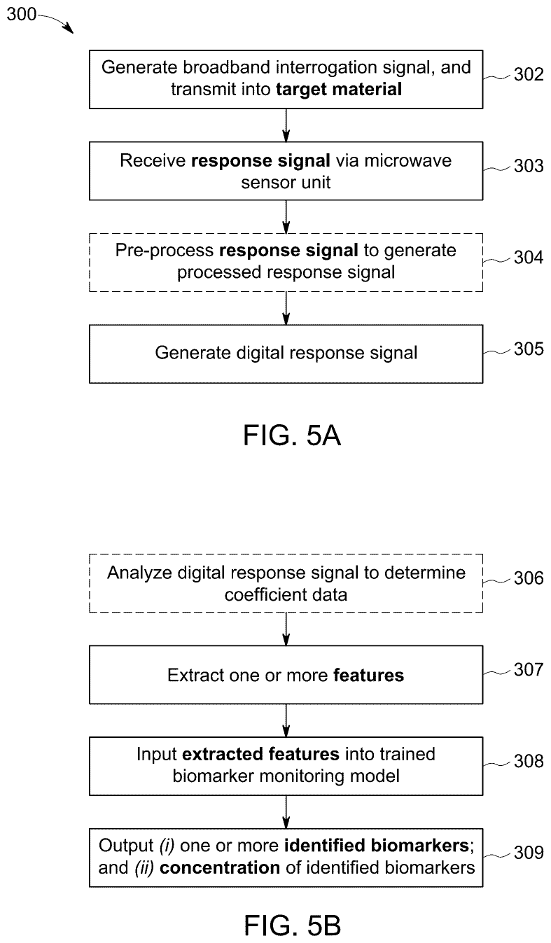

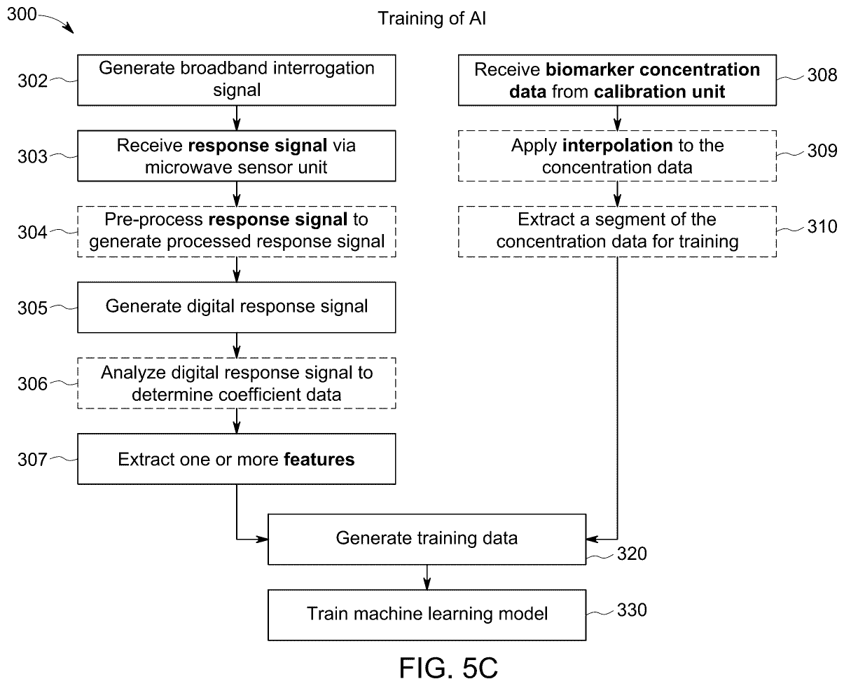

Accordingly, as can be seen with respect to FIG. 5A, the biometric detection and sensing device of FIG. 1, can be used to generate an interrogation signal at high frequency, such as at a range of microwave frequencies. Specifically, FIG. 5A sets forth a method 300 for employing the present non-invasive biometric sensing and monitoring device 1, such as in a method for determining a characteristic of a state of a user, for instance, based on a value of a biomolecule within a body tissue of the user. First, at Step 302 the biometric sensing and monitoring device 1 can be positioned on a body of the individual user 100, such as on the user's arm, as depicted in FIG. 3, such as by threading the arm through a pair of straps, tightening the straps to securely attach the device, and engaging an activation mechanism to begin the implementation of a sensing regime.

Upon activation of a sensing regime, a broadband interrogation signal may be initiated, such as by activation of a signal generator 8, e.g., frequency synthesizer. Hence, this activation generates an interrogation signal, at a specified frequency, which is applied by the frequency synthesizer 8 to port 1, 13a, of the microwave structure 12 at constant voltage and amplitude. For example, a starting interrogation signal may be applied at 100 MHz or less. This may be applied as a first iteration of the sensing regime. Then after propagation of the transmitted signal from pole 1, 13a, to pole 2, 13b, at Step 303, the signal response can be received from the microwave structure 12 of the microwave sensor unit.

Particularly, with regard to signal transmission, the signal can be passed along a microwave structure 12 in four different manners. The transmission dynamics of this signal propagation is an important feature of the microwave sensor unit 10 because, as explained herein below, it is by the manner in which the signal propagates from the first pole to the second pole whereby one or more properties of a biomolecule, or other component within a dielectric media in proximity of the microwave structure 12, such as the tissues of a living body, can be determined. For example, first, the applied signal produces an electromagnetic wave. A portion of the electromagnetic wave may propagate in a confined manner along the microwave structure in a forwards direction from port 1, 13a, to port 2, 13b. The energy from this transmission is designated. S21, and it remains roughly constant regardless of the biomolecule being tested for and/or monitored. This energy may be detected as direct transmission at port 2 by the power detector 20.

A second a portion of the electromagnetic wave of the applied signal may propagate in a confined manner along the microwave structure in a forwards direction from port 1 toward port, but after traversing some distance towards port 2, the energy from this signal may then reverse course and be backwards propagated back to port 1. The energy from this reversal of transmission is called reflectance energy, and it is designated as S22. The reflectance energy can be measured at terminal 1 by a return power detector unit, such as a circulator 21.

The third and fourth portions of the electromagnetic wave of the transmission signal may be non-confined and, therefore, may propagate outside of the microwave structure, such as between the microwave structure and the ground plane. These depend on the design of the microwave structure as well as the dielectric medium proximate the microwave structure. In these regards, part of the electromagnetic wave will get transmitted through the dielectric medium, such as in the form of fringe fields, and will make it from pole 1 to pole 2 to be received and be detectable at Step 303 as part of the transmission response. However, another, smaller, part of the non-confined signal will simply radiate away and be lost.

Hence, at Step 303, there are two parts of the signal that make it all the way across from port 1, 13a, to port 2, 13b, of the sensor device 1, and these include the confined portion of the applied signal that propagates within the microwave structure 12, and the non-confined, fringe filed portion of the applied signal that propagates through the dielectric medium proximate the microwave structure 12, which in various iterations set forth herein may be a portion of the human body 100. In such instances, the non-confined fringe fields propagate through the environment between the microwave structure 12 and the ground plane 16.

For instance, as discussed above, as the interrogation signal is transmitted from the first port 13a to the second port 13b along the microwave structure 12, a plurality of fringe fields may be produced. As can be seen with respect to FIG. 4, once produced a plurality of the fringe fields will expand outward away from and circle back to the microwave structure 12, and in so doing will then interact with the tissues of the human body and the constituents therein, including a number of biomolecules. However, some of the energy released along with the fringe fields will be lost as radiation, and some will be lost through dielectric loss. These types of radiant energy are simply lost and cannot be accounted for.

Nevertheless, a large portion of the fringe fields will enter the environment of the body 100 proximate the microwave structure 12 and may traverse through the body from the first port 13a to the second port 13b of the microwave sensor unit 10. This type of fringe field can penetrate deeply into the environmental media of the body, such as into the various tissues and interstitial fluids and other components of the skin. These fringe fields, therefore, are affected by all of the elements, e.g., within the tissues, with which they come into contact. The depth can be from microns to centimeters. At lower frequencies penetration can be deeper, whereas at higher frequencies, such penetration may be more shallow. Regardless of the depth of penetration, and dependent on the frequency of the fringe fields, as discussed above, some of the energy will be absorbed by the various components of the skin, such as based on whether its dielectric constant corresponds with the frequency by which the fringe field was generated.

Consequently, at Step 303, these fringe fields can then be measured at the second, reception port 13b, such as in addition to the confined transmission energy. The overall energy transferred to the second port 13b is the transmission response which includes but is not limited to the contained energy and that of the fringing fields. For example, the primary component of the interrogation signal is the electromagnetic wave generated by the voltage applied at Port 1, 13a. This wave propagates through the microwave structure 12 towards Port 2, 13b. Fringing fields are part of the overall signal but represent only a portion of it. These fields extend beyond the physical boundaries of the transmission line 12 and can influence how the electromagnetic wave behaves, especially near edges and discontinuities.

Other factors can also affect efficient transmission. For example, there are transmission line effects that occur such as when the signal experiences transmission line artifacts, including reflections, losses, and impedance mismatches. These factors can affect the amplitude and phase of the signal as it travels from Port 1 to Port 2. Then there are also dielectric effects. For instance, the dielectric material, e.g., body tissue 100, surrounding the microwave structure 12 affects how the electromagnetic waves propagate. The dielectric constant influences the speed of the wave and can lead to dispersion, where different frequencies travel at different speeds. There is also interferences. Particularly, the interaction between various signals, including reflected waves and fringing fields, can lead to constructive or destructive interference, affecting the overall signal received at Port 2, 13b.

Nevertheless, despite these interferences and inefficiencies, a large portion of the non-confined transmission signal flows through the dielectric medium, between the microwave structure 12 and the ground plane 16, is received at port 2, 13b, e.g., as an analog signal, as explained below, and at Step 304, may be preprocessed, such as in a first pre-processing step, and be converted into a digital signal at Step 305. Hence, at Step 305, the pre-processed transmission response data may be converted to a digital signal, such as by the analog to digital converter 25, so as to produce digital transmission response data, which digital transmission response data can then be communicated to the analytics system 204, such as over a wireless, cloud-based communications network, as set forth in FIG. 4, so as to be preprocessed thereby in a second preprocessing step. Particularly, with respect to the non-confined, fringe field portion of the transmission signal, the interaction of the fringe fields with the human body, e.g., the skin thereof, is mediated by the dielectric permittivity of the skin. Likewise, the permittivity of the skin itself is arbitrated based on the various different biomolecules being present within the skin, such as with respect to the identity and quantity, e.g., concentration, of the biomolecules within the body tissues, e.g., skin.

As set forth in FIG. 4, exemplary biomolecules that may be detected and characterized include glucose, ketones, alcohol, lactate, and the like. More specifically, as set forth in FIG. 5B, the interaction of the presence of the biomolecule within the body tissues, e.g., skin, affects the permittivity of the skin, which permittivity then affects the ability of the electromagnetic field wave to traverse the microwave structure, such as in a concentration-dependent manner. The dielectric effects of these various biomolecules on the propagation of the fringe fields through the body, as explained herein, can be used in a method for identifying such biomarkers, as well as for determining their concentration.

More particularly, with reference to FIG. 5B, presented therein is a follow on method to that of FIG. 5A, wherein the digital response signal generated at Step 305 may be transmitted to the analytics module 50 of the system 200, whereby the digital response signal may then be analyzed in accordance with the method set forth in FIG. 5B. For instance, the digital response signal produced in Step 305, may be further processed and analyzed so as to determine the presence of one or more biomolecules within the tissues proximate the biometric sensor unit 1 as well as their concentration, such as by analyzing the manner by which the non-confined, fringe field portion of the interrogation signal propagates through the body tissues.

The presence of various biomolecules within the body tissues 100, e.g., skin, affects the speed of transmission along the microwave structure 12, when that structure 12 is placed in proximity to the skin. The rate at which the fringe fields propagate through the tissues of the body depends on the surrounding environment and the dielectric permittivity of the biomolecules as well as the frequency of that signal. As indicated above, permittivity is a fundamental property of materials that measures how an electric field affects and is affected by a dielectric medium. A higher permittivity results in a slower signal speed due to increased capacitance and reduced propagation velocity. This can be seen with respect to the presence of glucose within the body. For instance, the dielectric permittivity of a glucose solution is less than that of water, so an increased concentration of glucose results in a reduction of dielectric permittivity, and therefore, increases the speed of microwave signals propagating through the skin, which can then be detected at port 2, 13b, of the sensor unit 1.

Consequently, the overall permittivity of the skin, or any other tissue, will be based on the biomolecules being present therein, and thus, based on these permittivity effects, those biomolecules may be characterized, as set forth in the method of FIG. 5B. Specifically, as the part of the passing transmission signal propagates through the body tissues 100 in the form of fringing fields, their interaction with the various components, biomolecules, of the tissues beyond the microwave structure 12, affects the speed of transmission, such as based on the dielectric permittivity of the biomolecules that interact with those fringe fields at the emitted frequencies. Particularly, due to the dielectric permittivity of the human body at a range of high frequencies (e.g., from about 100 MHz to about 10 GHz), the speed at which the referenced fringe fields propagate through the body is going to be dependent on the identity and concentration of the biomolecules being present therein, such as with respect to their respective dielectric permittivity and their effects on that of the body. This speed of fringe field propagation, therefore, will change as the environment of the body tissue changes. In these regards, the higher the overall dielectric permittivity, the slower will be the propagation speed.

Further, as indicated, the dielectric materials of the human body, also impacts the field in terms of dielectric loss. Each dielectric material not only has frequency dependent permittivity response, but also produces a unique dielectric loss. This means that in addition to the change in propagation speed, it also changes the lost power of the microwave signal. All these parameters change the received signal at port 2, 13b, of the microwave structure 12.

When the concentration of biomolecules in the human body changes, e.g., at a given frequency, all of the above referenced parameters also change. These changing parameters result in changing the measured output, e.g., amplitude, at port 2, 13b, of the microwave structure 12, dependent on their correspondence with the transmitted frequency. In particular implementations, a number of these variations can be sensed and measured via a resonant sensing mechanism, and their effects can be accounted for by the analytics module of the system, such as in consideration along with the detectable transmission response. Measurement of these variations is difficult to characterize and gauge because they do not follow the more discernable pattern as the referenced fringe field distribution patterns described herein, such as on a frequency to frequency basis.

However, given the presence and effects of the referenced fringe fields on the body, the transmission response may include the measurements of these fringe fields as well. In such instances, the overall transmission response may be based on both the transmission (S2 measured at the power detector 20 at port 2) of the current through the microwave structure 12, as well as the fringe field arriving at the reception pole 13b, but may also include the measurement of the reflection signal (and/or fringe field) arriving back at the transmission port such as due to the reflection processes (S1) described herein, and may further account for the aforementioned losses described above. Nevertheless, although all of these various measurements may be analyzed and used in the various determinations described herein, such as via analysis by the artificial intelligence module, as described herein below, in various embodiments, a very useful measurement will be the detection and determination of the fringe field data arriving at pole 2, 13b of the microwave sensor unit 10.

Hence, in various embodiments, the overall transmission response may be based on both the transmission (S2—Measured at the Power Detector 20 at port 2) of the current as well as the fringe field arriving at the reception pole, but may also include the measurement of the reflection signal arriving back at the transmission port such as due to the reflection processes (S1) described herein. Therefore, the overall transmission response may be the sum of the direct transmission signal in addition to the received fringe fields, and may further account for the reflection signal as well as the various aforementioned losses, such as including the line loss, dielectric loss, fringe field losses, and various other inefficiencies, and the like. Accordingly, given the presence and unique permittivity effects of the referenced fringe fields on the body, the fringe field transmission response may be a particularly useful measurement being made and used to determine the identify and concentration of biomolecules being present within the tissues of the body, without reference to the measure direct transmission response and regardless of the various losses encountered.

In any of these instance, the overall transmission response, or a subset thereof, may be determined by the power detector 20 and may be the sum of one or more of the direct transmission signal in addition to the fringe fields and may or may not account for various aforementioned reflection signals and losses. For instance, as indicated, in various embodiments, the measurement of the fringe fields propagating through the body 100 proximate to the microwave structure 12, and between the ground plane 16, is an important feature of the system and how t works that can be used to demarcate and characterize biomolecules within the tissues through which the fringe fields are propagating. As indicated, the body tissues and biomolecules therein each have a unique dielectric permittivity.

Consequently, as the electromagnetic field passes through the body tissues and contacts the various biomolecules therein, if they have a frequency that corresponds with the dielectric constant of the biomolecule, the molecule will act as a capacitor and absorb a certain portion of the energy, thereby slowing the progression of the fringe field through the tissue and lessening its amplitude as it arrives at port 2, 13b, of the microwave sensor unit 10. Particularly, at this point, the power detector 20 connected to port 2, 13b, can measures this subset of the overall power received at each frequency, and can then measure and convert it to a low-frequency signal that can then be converted into a digital signal by the analog to digital converter 25. In this instance, the reduction in the measured amplitude of the fringe field as compared to the measured amplitude of the confined transmission can be distinguished, and used to identify one or more biomolecules of interest, where the drop in amplitude is presumed to correspond to the capacitance of the biomolecule, albeit summed over all biomolecules that resonate with that particular applied frequency.

As the frequencies change, the capacitive response of the milieu of biomolecules will change. Likewise, as the capacitive response of the biomolecule milieu changes, then the measured amplitude, e.g., the capacitive response, at port 2, changes. This capacitive response may be a measurement of the overall capacitance within the system, e.g., the microwave structure thereof, or a subset thereof, such as in comparison between the confined versus the non-confined transmission responses. Consequently, because the skin is a complex organization of dielectric components, its overall permittivity will be a composite of all the dielectric constants for each of these components. Therefore, by exposing the skin to a number of different signals of different frequencies, creates a large number of response data that can be correlated together in different manners so as to isolate the response to each particularized component.

In this manner, the resistivity, capacitance, and/or permittivity of the various different components of the skin, and of the skin, or other tissue of the body, may all be identified, quantified, and qualified, such as by performing a number of measurements as described herein. Consequently, the dielectric permittivity of the skin, as a whole, or one of its component parts, e.g., constituents, can all be characterized, e.g., finger printed. Thus, the permittivity of the skin is affected by the permittivity of its constituents, which is a reflection of the condition of that skin at that given time, e.g., due to its environmental makeup.

As the biomolecular makeup changes, the environmental milieu of the skin changes, as does its permittivity, and all of these characteristics can be defined and measured by the devices and systems set forth herein in accordance with the referenced methods. Specifically, this loss of amplitude is a unique electric characteristic or feature caused by the dielectric permittivity of each different biomolecule. Such dielectric features as this can be used to characterize and quantify the biomolecule.

However, it is possible that at any given frequency, there might be similar bonds within two different molecules that may cause those two molecule to act in a similar fashion in response to the passage of a fringe field at that particular frequency. This would, therefore, cause confusion as to what the identifies are of these two different molecules, because they are acting in a similar fashion. Therefore, to overcome this confusion, a number of different frequencies can be applied to the microwave structure 12 by the frequency generator 8, so as to generate a dielectric spectrum, or signature, for the various biomolecules of interest. This is useful because although two different molecules may act similar at one given frequency, the probability that they will act in a corresponding manner over a broad spectrum of frequencies is very low.

In view of this, the method set forth in FIG. 5A, can be repeated a number of times so as to characterize the various different biomolecules within the tissue being observed in response to all of a set of applied frequencies such as to determine the broad spectral response, e.g., dielectric fingerprint, of the respective biomolecules to that set of frequencies. These frequency based changes in dielectric permittivity changes the amplitude of the electromagnetic waves of the fringe field, which then causes a corresponding change in the capacitance as measured when those respective fringe fields arrive at port 2, e.g., as measured by the power detector 20.

This set of frequencies can be any number of frequencies but the number should be selected so as to be large enough to distinguish one biomolecule from another, and the frequencies can be applied in a number of sets, such as in an iterative fashion until the requisite number of frequencies have been applied so as to generate a sufficiently narrow vectorized characterization, e.g., fingerprint, for the biomolecule, e.g., over a wide spectrum of frequencies. The range of frequencies can be any variable range, but of particular usefulness will be a set of high frequency signals, such as in the microwave range, such as from about 100 mHz up to about 10 GHz, and all subsets there between. By use of a range of frequencies a set of dielectric partitivities for each molecule within the tissue can be defined, which can all be correlated to define a biomolecule based on which frequencies the biomolecule responds to, e.g., by absorbing energy therefrom, and with regard to the change of amplitude that response provokes, e.g., based on just how much energy it absorbs at any given frequency, such as measured by a concomitant change in capacitance at Port 2. In this regard, each measured response, e.g., dielectric response, of a biomolecule to a fringe filed of a particularized frequency can be expressed as a vector.

Accordingly, after traversing the microwave structure, the transmission signal is received at the second, e.g., output, port, where it can be determined and measured, such as by the readout circuitry. Specifically, after electromagnetic field generation and the initiation of transmission, an electromagnetic wave or signal traverses from a first, transmission port, to a second, reception port 13b. This process is termed herein as transmission.

Upon receipt at the second port, 13b, the signal can be read by a power detector being coupled therewith. Particularly, the power detector may be configured to measure the amplitude of the high frequencies being traversed along the microwave structure 12 at port two 13b. The power detector 20 then measures the amplitude at the respective high frequency and then steps it down to a lower frequency signal. This stepping down is useful, because the digital converter 25 works better at lower frequencies, because very high-frequency analog to digital converters have very low resolution, have high power consumption, and are very expensive. In addition, at each time the power detector primarily measures the output power at port 2 for a known frequency, so the information measured by the power detector is focused on the amplitude. Hence, the power detector 20 measures received voltage and frequency and can compare to applied voltage and frequency to determine difference, also want to measure amplitude, which amplitude may be measured, using an envelop of the signal algorithm

Next the lower frequency signal can be conveyed to the analog to digital converter 25 where the signal can then be converted, e.g., by the analog to digital converter 25 into a digital signal, such as a low frequency digital signal, which can then be fed into an analytics module of the system for further processing. This low frequency digital signal is the output of the power detector 20 and the ADC 25, which can then be communicated, such as over a wireless internet connection, e.g., WIFI, to an analytics module of the system 1, such as for further processing. In this regard, this measured digital transmission signal amplitude is of importance for determining the permittivity features of the various different biomolecules throughout the range of applied frequencies, and thus, each so measured amplitude may be encoded with metadata defining the conditions of the measurement so as to make correspondence with other measurements characterizing the same molecule at different frequencies easier to identify.

Specifically, as set forth with reference to the method of FIG. 5A, at each time, the device controller, e.g., the microcontroller 30, applies a signal to the frequency synthesizer 8. The frequency synthesizer 8 then generates a signal at a known frequency and that signal is applied to the microwave structure 12. The power detector 20 at port 2, 13b, receives that signal, and upon receipt of the transmitted and received signal, e.g., the confined and non-confined signal, is measured by the power detector 20. This is the power, e.g., amplitude, measured for that certain, e.g., first, frequency. The measured power at port 2, 13b, is termed the transmission response at this work. Particularly, the measured amplitude of the transmission response at each of these frequencies may be referenced as a feature, e.g., a dielectric permittivity feature.

Accordingly, this process is continued until all the frequencies of a selected set of frequencies to be tested are examined, their respective power measurements are recorded, their voltages and voltage differences are determined, their respective features are defined and converted into a number of vectors. The results of these analyses is a number of features together which form a feature observation, for a set of applied frequencies. For instance, where 25, 50, 75, 100, 150, 200, 250, up to 500 transmissions are applied, then the resultant feature observation will include respective feature amplitudes, as measured at port 2, together, all of which form a broad dielectric spectrum that can be analyzed in accordance with the methods herein so as to identify a number of biomolecules within the tissue under observation as well as to determine their respective concentrations.

Having a broad dielectric spectrum by which to determine and characterize biomolecules, e.g., based on how the presence of those biomolecules affect an applied fringe field, such as by their change in capacitance in the presence of that fringe field, is useful for characterizing those biomolecules. This is because any number of biomolecules may respond in a similar fashion to any give applied frequency, e.g. based on the similarity of their chemical structures, but it is unlikely that two different biomolecules will respond in a similar manner over a broad range of applied frequencies. Consequently, as determined by the inventors hereof, the generation of a broad dielectric fingerprint for each biomolecule is particularly useful for identification and concentration determinations.

Therefore, once the first applied signal has been received and measured at the power detector 20 and is processed by the analog to digital converter 25, as described above, the measured response signal may then be transmitted to the system controller 30, which response can be saved in an onboard memory of the system 200. This process may then be repeated, by the frequency synthesizer 8 generating and applying a second, third, fourth signal, etc. to the microwave structure 12, such as where each subsequent signal may be different than the previous. The sequence can be additive, reductive, or random, and can be applied in response to a sequence, number, generator that may be programmable software being run by the microcontroller. In a typical set of sequences the number of generated frequencies can be 5, 10, 20, 50, 75, 100, 125, 150 frequencies, or more, including the numbers in between.

The frequency generator 8 may apply any number of frequencies to the microwave structure 12 so as to produce a set of interrogation signals to be applied to the microwave structure, such as 250, 500, 1,000, up to 10,000 or more. The returned response signal for a set of frequency determinations is termed herein as an observation. The frequencies to be applied will typically be in the very high frequency, e.g., microwave, range, such as from about 150 MHz to about 1, 2, 3, 4, or 5 up to about 10 GHz, which can be applied in any number of sets, so as to produce a wide continuum of applied frequencies and thereby generate a broad spectrum of transmission response, e.g., measured amplitude, data.

As indicated above, each measured response, e.g., determined amplitude, corresponds to a dielectric primitivity of one or more given biomolecules being present within the environment that is proximate the microwave structure 12. Hence, the measured amplitude can be referenced as a single dielectric permittivity feature. Since the dielectric permittivity features of different biomolecules are different for various frequencies, by measuring the output at port 2, such as by initiating and transmitting a large number of applied frequencies, a broad transmission response, encapsulating the multiplicity of applied frequencies, will be obtained.

Further, as disclosed herein, each feature can be converted into a one dimensional array and be expressed as a vector. Likewise, any two or more dimensions of these vectors can be combined to form a matrices. And a larger number, e.g., all, of these vectors can be combined to produce a vectorized observation, which may be characterized by respective applied transmission power, voltages, frequencies, and amplitudes, as well as received transmission response power, voltages, frequencies, and amplitudes, along with their respective differences between them.

This broad frequency response spectrum can then be used, not only to identify different biomolecules being present within the tissues, but to also quantify them, such as with respect to their concentration therein, e.g., based on the set of their applicable dielectric permittivity features. Specifically, the broad frequency spectrum, e.g., observation, for one biomolecule, e.g., for water, or glucose, or lactate, or ketones, and the like, will be different than the observation for the other biomolecules.

Accordingly, the analytics module 50 of the system can receive the transmission response, e.g., amplitude, data, measured at port 2, 13b, over a broad range of applied frequencies, and implementing a number of multi-dimensional problem solving algorithms, can correlate the applied frequencies to the measured amplitudes of the transmission response, and thereby define the dielectric permittivities of the various biomolecules predicated to be present within the tissue. Particularly, this data can be used to build a spectral map for each biomolecule, such as by employing a large knowledge base data structure, whereby a number of dielectric feature vectors can be defined and associated with one or more biomolecules.

These vectors can be assigned values corresponding to the set of biomolecules to which they may be associated, such as based on the unique dielectric permittivity response of the biomolecule to the fringe fields of one or more frequencies, and the sum of feature vectors for any given biomolecule can then be compiled, so as to produce a dielectric permittivity fingerprint, which can then be used to define the various different biomolecules being present within the tissues. In such instances as this, each individual feature can represent a measurement of at least a portion of the power received at port 2, 13b. In particular embodiments, the feature represents the power differential between the amplitude of the applied power minus the confined transmission power versus the amplitude of the non-confined transmission measured at port 2, which can be expressed as the ratio of the non-confined power divided by the confined power, at each applied frequency. As indicated, since the response to each frequency provides one feature, and since a multiplicity of frequencies, e.g., 150 different frequencies, may be applied over time, the resultant raw fingerprint data may have 150 features that all depend on the dielectric characteristics of the human body 100 at the ambient of the microwave sensor 12.

For these purposes, as set forth in FIG. 5A, in a first step, a first frequency, such as at 100 MHz, may be generated and transmitted along the microwave structure, and a first set of transmission measurements may be determined and/or analyzed. Next, this transmission process may be repeated for other frequencies, such as 120 MHz, 150 MHz, etc. up to 10 GHz and beyond. This repeating of multiple transmission processes over a broad spectrum of frequencies is useful, such as for distinguishing between biomolecules, because at any one given frequency, the permittivity features of a frequency transmission scan can be similar for a number of biomolecules. Particularly, the response of the body to a received fringe field in the presence of a first biomolecule within the environment of the body's tissues may be similar to the body's response to a second biomolecule, such as when the frequencies are the same or similar. However, the body's permittivity response over a multiplicity of frequencies in the presence of the two referenced biomolecules is going to vary widely. Consequently, subjecting the body, via the presence of the microwave structure, to a number of different frequencies, e.g., a wide frequency range, produces a broad spectrum response that is as distinguishable for each individual biomolecule that may be as unique as the body's fingerprints.

Thus, in accordance with the methods herein disclosed, the presence and concentration of a biomolecule within the tissues of a body can be detected, determined, and measured by the tissues unique permittivity fingerprint-like response. Specifically, it is expected that the dielectric permittivity spectrum response over a broad range of frequencies, e.g., permittivity fingerprint, will not be the same for any two biomolecules within the body. However, it is expected that such responses will be similar between different bodies, thus, allowing for the present system to determine the presence and concentration of different biomolecules in a systematic manner based on each body's uniformly characteristic broad permittivity response spectrum, e.g., based on the correspondence amongst broad spectrum permittivity features that are common for the same biomolecules across different bodes.

This repeating of the transmission and measurement processes is useful because the body will have different permeabilities based on different transmission frequencies. Specifically, the body's natural permittivity changes over a broad range of frequencies, thus providing a spectrum of different responses to the different frequencies, which is further differentiated based on the various different concentrations of biomolecules within the tissues forming the transmission environment around the microwave structure. Thus, there is a complex relationship between the body's response to a variety of electromagnetic waves of different frequencies being applied in proximity to the body's tissue, which relationship is modulated by the presence and concentration of the various biomolecules being present within the tissues, and/or the interstitial fluids therein. This relationship, e.g., based on the fact that the permittivity of the tissue changes in response to the frequencies of electromagnetic fields being applied in proximity thereto, can be employed, as described herein, so as to determine the concentration of one or more of those biomolecules within and around the tissue. This relationship can be termed herein as a dialectic response, and the corresponding transmission response can be measured in relation thereto, e.g., at each respective frequency.

Specifically, as introduced above, as transmission begins, there are actually a main transmission response, which involves the movement of an electrical impulse, e.g., the propagation of an electromagnetic radiation wave, along the microwave structure. This is the primary transmission signal that gets received at the second, reception port. The propagation of this impulse along the microwave structure produces an electromagnetic field that travels linearly forward along the path of the structure to the grounded structure or port. However, there is also a plurality of other electromagnetic fields that get propagated, such as the above-referenced fringe fields. Subjecting the tissue to a broad spectrum of frequencies creates a range of dielectric responses, which responses are dependent on the type and number, e.g., concentration, of biomolecules within the transmission environment. Thus, the presence and concentration of biomolecules affect the permittivity of the body tissue proximate to the sensor, which permittivity affects the depth of penetration as well as the transmission of the propagation of the fringe field from the positive to the negative ports of the sensor.

As indicated above, the generation of a single vectorized observation for a given biomolecule is a computationally difficult task, and thus, in various embodiments, the analytics module 50 of the biometric sensing system may instantiate or otherwise employ one or more artificial intelligence modules for determining one or more features, vectors, observations, etc., for one or more biomolecules, using that information to generate a large data structure, and then employing that data structure to perform one or more calculations and/or comparisons of the data therein so as to determine the identity and characteristics of one or more biomolecules being present within a tissue under observation.

In this regard, as can be seen with respect to FIGS. 5B-5C, the analytics system may be configured for implementing one or more artificial intelligence modules 52 for the purpose of generating a model, e.g., via a suitably trained AI module, e.g., machine learning model, and then applying that model, as described herein, such as by a suitably trained inference engine, so as to identify the various different biomolecules being present in a sample of tissue, as well as for further determining their concentration and/or their effects on the human body. For these purpose, the collected and processed, e.g., pre-processed, data may be conditioned and/or be preprocessed prior to being transmitted to the analytics system 50 for further processing, such as for the performance of one or more data processing steps. Particularly, the biometric sensing and monitoring systems and devices set forth herein are configured for generating a number of feature, vector, and observation data for a large number of readouts, e.g., tens, hundreds, thousands, tens of thousands, hundreds of thousands, millions, and the like, over a broad spectrum of frequencies, so as to characterize any number of biomolecules within a tissue under observation. Likewise, all of these data can be collected and analyzed over one or more, e.g., a large number, of samples or sample sets, such as for a large number of people.

As will be readily understood, building and training an AI model, implementing a large number of features and observations, for a large number of people is a very difficult, computationally absorbent task. Building and populating the requisite data structure is very complex. As described herein below, any number of data structures may be implemented, such as a data tree, knowledge graph, nearest neighbor graph, artificial neural network, and the like, but in all cases, the implementation of the data structure will be complex, because there are a large number of known coefficients, e.g., measured data, as well as an even larger number of unknown variables to be calculated and/or adjusted. Further, all of this data, and its concomitant complexity, is made even more complex in their relationships when used to train and generate one or more models, such as an individual model that may be used to characterize each individual biomolecule of interest, such s for glucose, lactate, ketones, alcohol, and the like.

To help lessen the computational load, even though the initial measurements may include all readouts of a set of an observation, not all vectors, e.g., features, are going to be useful in generating a final characterization of the biomolecule. Particularly, what is of particular use for assessing any given biomolecule within a person, is really just the data related to measurement dynamics. What this means is that the analytics system is particularly interested in feature data wherein the biomolecules of interest actually had a change in dielectric response at a particular frequency. In those instances where the frequency applied did not result in a loss of power transfer, e.g., the amplitudes between the confined and non-confined transmission responses are largely corresponding, then the data for this frequency is not necessarily that important, because, since there was not a significant change in applied to received amplitude, it is likely that the biomolecule was not responsive to the propagated fringe fields at that frequency. Thus, for this particular biomolecule, at this applied frequency, the vector data is not of particular use for determining a characterization of this biomolecule.

Accordingly, as can be seen with reference to FIG. 5B, to lessen the computational load, a feature selection process may be implemented to cull the data used to build the data structure of that data that is not going to be significant to the ultimate determinations and characterizations. For instance, when generating a data structure, such as a neural network, e.g., an artificial neural network, the number of features to be considered can be filtered to exclude all features that did not show an appreciable change in power between the confined and non-confined transmission rates and/or amplitudes. These data may be excluded because where there is no appreciable change in speed and/or of power of transmission, it is assumed that the lack of differentiation between the two results is due to the fact that the biomolecule of interest was not appreciably permissive to that frequency.

Thus, the feature data pertaining to this frequency can, but need not, be considered in generating a characterization for this particular biomolecule of interest. For example, where the observation set includes about 150 features, the appreciable list of features can be culled down to an initial sample set of 5 or 10, or 15 or 20 or 25 or 50 or more features to initially be considered. In this regard, the system itself or a user thereof can set, or otherwise determine, e.g., automatically and/or autonomously, the number of features to be considered as significant.

For example, in accordance with FIG. 5B, a number of conditioning and/or pre-processing steps can be performed such as for feature selection, as well as for data preparation, prior to, or in conjunction with, building a data structure that can be used to generate one or more models for use in determining a plurality of characteristics for a multiplicity of biomolecules in a very complex environmental milieu, such as within a body tissue. Specifically, at Step 306 once the aforementioned measurements have been taken, such as at pole 2, 13b, the measured data may be pre-conditioned, as disclosed herein, and can be converted into a digital signal. Then the digital response data can be communicated to an analytics platform 50 of the system 200, and the conditioned digital data can be converted into pre-processed data that can be subjected to one more feature selections steps.

Particularly, as discussed above, a number of observation data may be collected, where each observation includes a number of features, where each feature contains a measurement, such as an amplitude measurements, e.g., an amplitude ratio measurement. For example, where the feature measurement was for a set of frequencies, such as 50, 100, 150, 200, 250 frequencies or more, the effects of these frequencies as applied to a microwave sensor unit 10 placed on a body tissue, the presence of one or more biomolecules within that tissue being observed, can be determined in accordance with the methods disclosed herein. In this instance, the sensor unit 10 can be used to generate a number of features, where each feature includes an amplitude measurement, e.g., amplitude differential measurement, and the set of measurements form an observation.

These observations, at Step 307 may then be subjected to a feature selection process, whereby the number of features to be considered and analyzed per observation is reduced. For instance, the number of features under consideration can be subjected to variance analysis, such as where the amount of variance for each feature measured over time. In such an instance, the number of features to be considered can be reduced to five, ten, twenty, thirty or more features, such as based on the degree of variance between the features. Specifically, where an observation includes 150 features, based on a performed variance analysis, those features to finally be considered can be reduced down to thirty. This variance analysis can be performed in a number of intervals, such as six intervals of five features, or vice versa.

For each interval, the feature with the greatest variance, such as indicating the greatest permittivity change due to the response to the fringe fields at that particular frequency, can be selected as a member of the 30 features to be analyzed in that respective observation. In this manner, for each observation, at each level, only those features with the highest variance will be included in the final observation analysis. Consequently, once culled down, the final observation may be subjected for further processing will include about five to ten to about fifteen highly variant features.

Particularly, in one example, 30 intervals of five or six features are analyzed with respect to determining the one feature with the highest variance. Therefore, within each interval, the variability of each feature is measured, and the feature with the highest variability is selected to be evaluated from each interval. Hence, after this process, the 30 features with the greatest variance can be selected as part of the final observation to be analyzed.

As indicated above, another manner by which computational complexity can be reduced is by modulating the building of the data structure, in this instance, the neural network so as to reduce the number of hidden layers and/or neurons per each layer, within the data structure. Specifically, with respect to minimizing the network generation process, to further lessen computational complexity, the number of layers, e.g., hidden layers or neurons, within the neural network can initially be set low, and more neurons can be added until the system is able to generate a significantly relevant results data. In this regard, to generate a suitably configured neural network, a given number of features can be selected for analysis, and a determined number of network layers can be instantiated.

For example, initially the network can be built in accordance with an radial basis function (RBF) to produce and RBF network, as described below, which RBF network may be instantiated with a single hidden layer, and that hidden layer could have as many neurons as is efficient to implement, such as 1, 2, 3, or 5, or 10 or more. For instance, a first layer having some number, e.g., 7, hidden neurons may be initially employed. The test net can then be run with regard to a number of features, and it may be determined whether the network hits the designated mark. If not, then the number of features, neurons, and/or layers can be increased, such as from 1 to 2 to 5 to 10 to 20 to 30 or 50 or 100 or more, and be tested again and again until the determined mark is reached, and then the approved network can be instantiated, the model can be trained, and then be used to determine results from the unknown sample sets, as described herein below.

In either instance, in accordance with Step 308 the extracted feature data may then be subjected to network training, and if the results obtained are determined to be statistically significant, the configuration can be stopped, the model can be generated, and applied in the referenced analyses, and at Step 309, when used in the performance of a bio detection and monitoring operation, one or more biomolecules of interest may be identified and their concentration determined. However, if the results in this configuration are found to not be statistically significant, then one or more of the number of features and network layers and/or neurons may be increased, and the training can begin again.

Particularly, as set forth in FIG. 5C, for training the model, as explained herein below, the collected data may be separated into two different data groups. One proportion of a set of data can be classified as training data, and may be used to train the model. A second proportion of the set of data can then be used as a validation set of data, which the machine learning module has not had access to, and thus, can be applied to the generated model and used to validate the model. In both of these instances, the data from each of these data sets, can be known data, such as where the results are known. Thus, once an initial model is generated, e.g., using a test set of known results data, then the generated model can be presented the known validation data, where the machine learning model does not have access to the known results, and the model can be applied to the validation data set, and a set of results can be called. In such instances, the system generated results derived from applying the model to the validation set, can then be compared to the known results, and if the two result sets are substantially the same, then the model can be applied to unknown data and the answers can be used to characterize the biomolecules of interest.

However, if the validation results do not significantly match the known results data, then these steps can be repeated, changing the system weights, e.g., coefficients, and retaining the model, e.g., a number of times, until the two results sets are determined to be significantly the same, e.g., based on derived, or otherwise known, convergence criteria, such as having known mark value (about 4% or 5% to about 10% mark value) for training versus a corresponding known mark value (about 4% or 5% to about 10% mark value) for validation. These data can then be used to create the neural network, as referenced above, with the appropriately apportioned weighting. Particularly, if the achieved mark is more than the defined mark value for training, then the various factors can be re-weighted, more features and/or layers may be added, and the training regime can be repeated and repeated again, until the sufficiently defined mark value is achieved. Once the network has been created, and sufficiently trained, such as by the machine learning engine 53, then the model may be applied to new, unknown measured data, such as by the inference engine 54, and new glucose concentrations can be estimated and monitored.

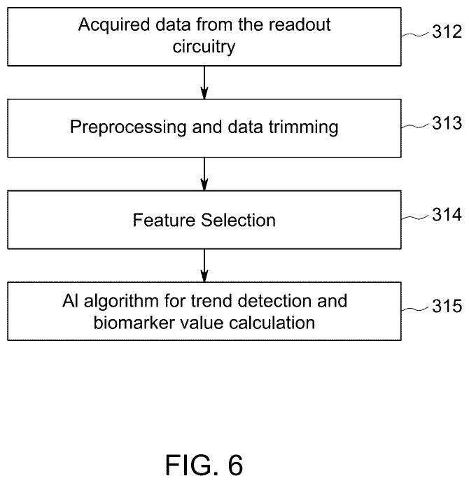

In this regard, in accordance with FIG. 6, in developing a model, e.g., AI generated algorithm, for determining the presence and concentration of a molecule within a tissue of the body, at Step 312, as described herein above, using the biometric monitoring and tracking sensor devices 1 herein, acquired measurement data may be generated by the readout circuitry 20 of the sensor unit 10. This acquired data may then be transmitted from the transportable wireless sensor device 1 to the remote analytics system 50. At Step 313 the acquired and transmitted data may be pre-processed, in accordance with the methods herein, and the data may be trimmed, as in FIG. 5B. In accordance with this process, at Step 315, a number of features may be selected for analysis, and the selected measurement data for the selected features may be applied to the generated model and a biomolecule concentration value may be calculated. This result may then be transmitted to the sensing, monitoring, and tracking device, or other mobile computing device, of the user, such as for display thereby, e.g., via a capacitive sensing display unit of the device 1, for the monitoring and tracking of the wearer's glucose, or other biomolecule, values. However, in order to achieve accurate and timely measurement results, the AI generated model, as indicated above, must be sufficiently trained to achieve a desired accuracy limit.

Accordingly, in one aspect, provided herein is a method for generating, training, and implementing a model by which unknown measurement data may be evaluated with respect to the model, and from this evaluation, a value, e.g., concentration, of a determined biomolecule within a tissue may be accurately predicted. For Example, as indicated above, a large number of data, such as measurement data, from a large number of observations may be collected in accordance with the methods disclosed herein. However, a simple computational system for analyzing such complex data, whereby a plurality of characteristics for a multiplicity of biomolecules, in a very complex environmental milieu, such the human body, need to be determined with a number of unknown variables, is too complex a process to be performed by generic computing means. To overcome these drawbacks, the present system may include, or otherwise access, an artificial system 52, which may be composed of a machine learning engine 53 and/or an inference engine 54. Consequently, in one aspect, provided herein is a method for producing, training, and implementing an artificial intelligence system 52, such as for use by the devices and systems of the disclosure in one or more methods for determining a characteristic of a state of a user based on a value of a biomolecule within a body tissue.

For these purposes, provided herein are methods for generating one or more models by which the presence and/or concentration of a biomolecule within a body tissue may be determined, such as by placing the non-invasive biometric sensing and monitoring device of the disclosure on that body tissue, and collecting a plurality of measurements therefrom. The model, once generated, may then be employed to not only determine the presence and concentration of a biomolecule within the tissue, but may also be used to track the concentration of that biomolecule over time, as well as to provide insights as to the body's response to that concentration over time. In particular iterations, the system can monitor and track any number of biomolecules within the tissue, either individually or collectively, across time, including glucose, lactate, alcohol, ketones, and the like, all of which can be tracked, analyzed, and health related analytics can be used to generate insights as to the health of the wearer as well as to provide recommendations for how to better live in view of those insights. In one embodiment, the biomolecule to be tracked is glucose, the monitoring may be for the purpose of tracking blood glucose levels over time, and the insights to be generated may pertain to the health choices of the wearer, such as with regard to their dietary and/or exercise decisions.

This collected data may then be used, such as by a machine learning module 53 of the system 1 so as to generate one or more models by which the identity and/or concentration of a biomolecule within a body tissue may be determined. Particularly, in training the model, as set forth with respect to FIG. 5C, there are two types of measurement data being collected. The first type of data is unknown data. The unknown data, as herein described, is the measurement data obtained by the bodily attached biomolecule sensing and monitoring devices 10 described herein. This data is unknown because it is raw measurement data, and its relationship to any particular biomolecule, its presence and concentration, and/or effects within the body is unknown.

The second type of data is known data. Specifically, at Step 308, the system 200 may collect a number, e.g., a large number, of known measurement data. In this instance, with regard to generating a glucose monitoring model for use by the system 200 for determining the presence and concentration of glucose within the body, the known data may be known blood glucose levels obtained from an auxiliary blood glucose measuring device. With this regard, any accurate blood glucose monitoring device and methodology for collecting blood glucose level readouts may be employed. For instance, the known glucose monitoring data may be generated and provided to the system by a traditional finger prick blood glucose monitoring device, or any number of invasive, wireless monitoring system that generates known blood glucose levels.

It is to be noted with respect to the present explanation that although the biomolecule of interest is glucose, the same methodologies can be employed in a similar manner so as to determine a model for use in tracking other biomolecules, such as ketones, alcohol, lactate, and the like, as set forth in FIG. 4, with the appropriate modifications being made to the known testing and monitoring devices. Likewise, the system 200 can be used to further monitor and track overall health, including dehydration, and can analyze trends over time to generate one or more recommendations as to how to improve and maintain such health. Further, once one or more models have been generated, the methods may additionally include applying that model, such as by an inference engine 54 of the artificial intelligence system 52 of the disclosure, to the collected data so as to identify a number of biomolecules within the body tissue as well as for determining their concentrations.

Accordingly, provided herein is a method 300 for generating a model for tracking biomolecules as can be seen with reference to FIG. 5C. Particularly, in generating one or more of the referenced models, at Step 302 a plurality of measurement data, from a large number of test subjects can be collected and be stored within one or more databases associated with the system 1. At Step 303, this data is collected from all test subjects 100, each of whom is wearing the non-invasive, continuous biomolecule, e.g., glucose, monitoring devices 1 presented herein.

The measurement procedures are performed in accordance with the methods disclosed herein, such as with reference to FIG. 5A. Essentially, the sensing and monitoring device 10 may be placed on the body 100, and a number of iterations of measurements may be taken. In such instances, in generating the requisite data, at Step 302 the device 10 is activated, and a large number of iterative broadband interrogation signals are applied to the body. At Step 303 the measurements are taken, and the transmission response signals are collected, such as by the power detector 20 at port 2, 13b, of the microwave sensor unit 10.

Further, as disclosed above, in accordance with Step 304, the received transmission response signal can be conditioned in one or more manners, such that at Step 305 a digital response signal can be produced. For instance, the analog signal can be converted, e.g., by the analog to digital signal converter 25 to a digital response signal, the digital signal can be stepped down, and the digital signal, now at a reduced condition, can then be transmitted, via a wireless network connection of the system, to one or more analytics modules of the system. Once received by at least one analytics modules 50 of the system 1, at Step 306 the received digital response signal can be analyzed, by a computing unit 31 of the analytics system 50, and one or more, e.g., a plurality, of coefficient data, e.g., amplitude and/or amplitude ratio data, may be determined.

Furthermore, from this analyzed data, at Step 307, in accordance with the methods set forth in FIG. 5B, one or more features can be extracted. Specifically, with respect to FIG. 5B, at Step 306, as indicated above, the digital response signal can be analyzed to determine the referenced coefficient data. Then at Step 307 one or more features can be extracted, as described above, such as by identifying the amplitude, or a change in amplitude, represented by the decrease in the non-confined transmission response as compared to the confined transmission response, as measured at port 2, 13b, of the sensor unit 10, which is correlated to the dielectric permittivity of the amount of glucose being present within the tissue. After this, at Step 308a, the extracted features can be reduced, via the feature selection process described above, and/or at Step 308b, the features and associated transmission data, can be conveyed to the analytics system so as to be included in the data sets for training the biomolecule, e.g., glucose, monitoring model.

It is to be noted that a similar process can be implemented once the model has been trained, such as to actually derive biomolecule, e.g., glucose, concentration data, such as by initiating the inference engine 54 to access and apply the generated model to the received extracted feature data at Step 308. In such an instance, once analyzed, e.g., by the inference engine 54, at Step 309, the results, e.g., an identified and quantified biomolecule, such as glucose can be output along with its concentration. However, with respect to FIG. 5C, when generating the model, such as in a first instance, once the various features for the various observations have been selected, identified, conditioned, preprocessed, and/or subjected to variance analysis, then in accordance with Step 320 of FIG. 5C, the collected data may be separated into two different data sets for use in generating and training the machine learning model at Step 330.

Furthermore, as can be seen with respect to FIG. 5C, a further set of data can be collected an used to train the model, as set forth above, and this data represents known data sets, such as collected by one or more known measurement devices. Consequently, at Step 308, such known biomolecule concentration data can be collected and provided to the system 200 so as to be evaluated by the machine learning engine 55 for the purpose of training the model, for instance, in one or more calibration methodologies described herein. In such instances, the measurement data can be considered interpolation data, e.g., unknown data, which data can be used, along with the known data, e.g., a discrete set of known data points, so as to generate a data structure. From this data structure, the unknown measurement data points can be evaluated against the known data points so as to estimate inferred concentration values to thereby generate a model by which future concentration characteristics can be estimated by applying the model to future unknown measurement data, as described herein.

Therefore, at Step 309 the method may include applying the unknown, interpolated data, to the known concentration data, so as to train the model. For example, at Steps 310 and 320, the extracted unknown feature data can be used to estimate glucose values and make a prediction of an estimated glucose concentration call, which call can then be compared to the known glucose value, such as in a training process, in accordance with Step 330. As described herein, where the estimated call matches the actual known concentration value, the model can be validated and used by the system to evaluate future measurement data and make one or more glucose concentration, or other characteristic, calls. However, where the estimated call does not significantly match the known values, then the model can be reconfigured, new training regimes may be implemented, and the process can be repeated a number of times until a determined accuracy level is determined.

More specifically, in training or recalibrating the model, the interpolation, e.g., measurement, data set along with the known data sets can be divided into two or three groups, a training group, a validation group, and a testing group, and a series of training, validating, and testing regimes can be iterated so as to generate and develop the model, validate the model, and then test the model any number of times as required in order to achieve a working model that reliably predicts the accurate concentration of the biomolecule based on observation of the measured data, such as with a determined level of precision. In these regards, the testing regime can be performed in any number of different manners.

For instance, a selection of test and control subjects can wear a selection of test or placebo decoy devices for a selected time period. They can each wear the designated device and at periodic time points can measure their glucose levels by the true and decoy devices. Likewise, over one or more time periods, each subject may be given a known concertation of a glucose (or placebo) to drink, and measurements may be taken by both the known glucose measurement devices and the system devices.

For the true system test devices, the measured data may be transmitted to the analytics system 200 where the data may be evaluated in accordance with the methods herein described, e.g., the measured data may be evaluated against a generated model, and a predicted glucose concentration can be called based on the preliminary model. Where the called concentration matches the known concentration, within a selected range of accuracy, the model and its configuration may be given more weight. Where the called and known concentrations do not accurately match within the required accuracy range, the model can be reconfigured and re-weighted, and the new model can be subjected to a new set of the test data and a new call can be made and compared to the known data, and the accuracy can be determined.

These steps can be repeated until a determined accuracy level is achieved. In these manners, the test unknown data sets can be applied to the model being generated, and preliminary concentration calls can be made. Then these calls can be compared to their corresponding known concentration values, so as to evaluate the accuracy of the calls and therefore the accuracy of the model. For example, in various implementations, this testing can be performed in accordance with a radial basis function (RBF) so as to build out the initial network upon which testing may be performed, as introduced above.

More specifically, the initial network may be generated by use of a radial basis function (RBF). An RBF is a mathematical function that has a value that depends solely on the distance between an input point and a fixed “center” point. This means that only the distance, e.g., how far away a given point is from a reference point will be considered. This methodology is very useful in building networks, as described herein, because it provides approximating functions that can create smooth surfaces from scattered data points, such as where the value decreases as the distance from the center increases. Consequently, an initial RBF based neural network may be generated, such as where the RBF network only has one hidden layer, which can have any number of neurons, which in this case are the selected features, e.g., 5, 7, 10, etc.

Particularly, with respect to building the initial test network, which may initially be a radial basis function test network, a small number of extracted features, e.g., 15 or 10, or 5, or less can first be used as test data. The referenced starting features can be selected and extracted in accordance with the methods of FIG. 5B, and the results of which may be tested in accordance with the method steps set forth in FIG. 5C. Particularly, the selected features can be separated into training, validation, and test sets, and a convergence criteria having a marked value for training and validating the sets can be selected.

The mark value may be selected at any sufficient, statistically significant value, and it can, but need not, be the same for the training, validation, and tests sets. For example, the mark value for training may be at 4%, whereas the mark value for validation may be at 10%, etc. The training data set may then be used to create the initial network, and the various weighting of that data can be apportioned based on the known data sets. Consequently, the initial network can be used to generate a set of predictions as to biomolecule concentration levels, which can then be compared to the known concentration levels for that biomolecule, e.g., within the sample tissue, and a score can be achieved and compared to the selected mark value.

This initial training set gives a set of coefficients, and the network can be run and re-run on the training data upon which the network was initially built. If the mark is less than the defined value for training, then the validation data can be tested, which data has not been shown to the initial test network at this time. Consequently, the test network can then be run against the validation data. if the mark for the validation data is also less than the mark defined for the validation, then the generated network is good and can be run against the unknown data and the results can be used to determine and monitor biomolecule concentration, e.g., within the body tissues.

But, if the test fails to meet the criteria and the test net is over the mark, then the network will need to be adjusted, dependent on which mark was not hit. For instance, the training mark may be hit, but the validation mark may not, or both marks may be missed. In either instance, the number of neurons, e.g., features for evaluation can be increased to a selected set point, e.g. 30, new marks set, and the test may be run again. The number of neurons to be considered can be iteratively increased into the network becomes validated, e.g., the predictions based on the known test sample sets run accurately compare with the known results.

Accordingly, with reference to FIG. 5C, the training data is generated, which includes, for each time step, within a time interval: (i) the corresponding true or interpolated concentration value obtained from the calibration unit(s); and (ii) extracted features, generated from the response signal received at that time step. Generally, the time interval of the calibration unit may be much larger than that of microwave data interval captured by the sensor through interrogation. For example, the time interval for testing a given biomolecule, e.g., lactate, was about 3 minutes/sample per test via finger pricking, was while it was 4 seconds/sample for the measured microwave data. For this reason, interpolation may be performed to provide a pairing between both types of data, for each time step.

As indicated, the machine learning model is trained to correlate (ii) feature data, to (i) actual readings from the calibration unit. Hence, the trained model is able to identify and understand different biomarker patterns. This is useful because, as explained above, each biomarker has its unique, distinct dielectric signature, and therefore generates a unique impact on the sensor's response. These features are general and are the results of the presence of all the materials in the near-field region of the microwave sensor including the biomarkers.

In view of the above, the disclosed devices and systems set forth herein were tested and the results of such testing are provided below. In at least one example, the biomarker monitoring model is a feedforward neural network with 2 hidden layers. The model may be trained using a backpropagation method. The number of the input layer's nodes is 23, and the number of the neurons in the hidden layers is 31 and 9 respectively (for each of the two hidden layers), with over 50 epochs used for training. The feature selection was accomplished based on feature ranking with 23 most impactful features selected. The output layer calculated the concentration of the target biomarker. Many other machine learning models such as LSTM, RNN, CNN, RL, RBF, etc. may also be used.

To that end, the biomarker dielectric signature is not affected by the medium, but the overall dielectric permittivity spectrum of the medium impacts the response of the sensor. Since the human body is an extremely complex environment for microwave applications with many variables, the number of data points was enhanced. This provided the ability to train a machine learning model to analyze the response of the sensor.

In at least one example, the feature extraction is developed based on a custom-built genetic algorithm consider the orthogonality of various initial features. Different number of features is considered for training the model which were preprocessed using a custom-built radial basis function system.

In some examples, the extracted features, the training data, and the trained model are different for the different biomarkers. However, the sensor, the readout circuitry, and the overall frequency points or data points are the same for all the possible biomarkers.

Accordingly, it is believed that the trained biomarker monitoring model, which uses broad-spectrum multi-feature data consideration capability, allows for the detection and monitoring of subtle changes in biomarker levels across physiologically meaningful concentrations.

### Example Test Results

i. Lactate Monitoring

As an example of the successful implementation of the technology, a human trial was performed in which nine (9) individuals volunteered to participate in lactate monitoring. The participants went through a certain procedure approved by the Research Ethics Office at the University of Alberta, Canada.

Participants were expected to attend two laboratory-based sessions. The first of these was a baseline test. During this session, demographic, anthropometric, and health-related data were collected. This session also involved a strength test where the participant completed a 10-repetition maximum (10RM) protocol for the leg extension exercise.

The 10RM was determined with a two-legged leg extension exercise and the velocity of movement was set by metronome to allow for two (2) seconds for the concentric and 2 seconds for the eccentric movements. The range of motion was set to allow a range of motion of the knee angle from 90° to 170°. After the 10RM was established, it was verified after a 5-minute recovery period to ensure that exhaustion occurred with 10 repetitions.

On a separate day, at least two (2) days after the 10RM testing, the two-legged exercise protocols began with 10 repetitions at 30% 10RM as a warm-up. The protocols consisted of 3 sets with a 3-minute rest period between sets. Sets performed with the same velocity, range of motion, and resistance as used for the 10RM. If loads cannot be completed or are exceeded during one set, it was adjusted for the next sets.

The data from the microwave structure placed on the wrist of the participants was recorded at 10-second intervals during the whole experiment process. Blood lactate measurements from the capillary meter were taken immediately before (pre) and after (post) each of the three sets, as well as every two (2) minutes after the last set. For the capillary lactate, the finger was cleaned, dried with gauze, and pricked with a one-time-use lancet. After wiping away the first drop of blood, the second drop was analyzed using the Lactate Plus™ lactate meter as the gold standard for calibration and comparison purposes.

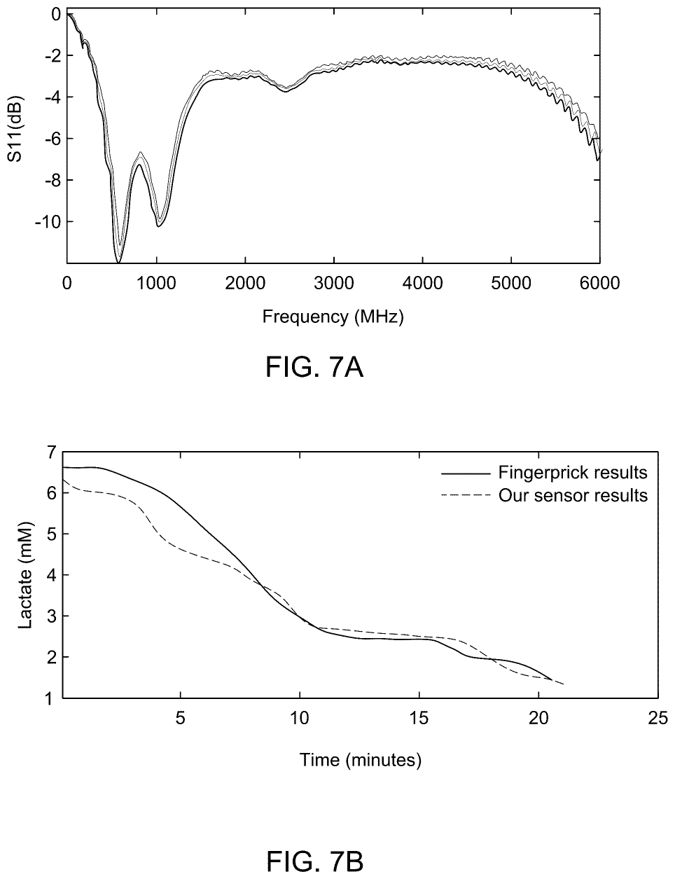

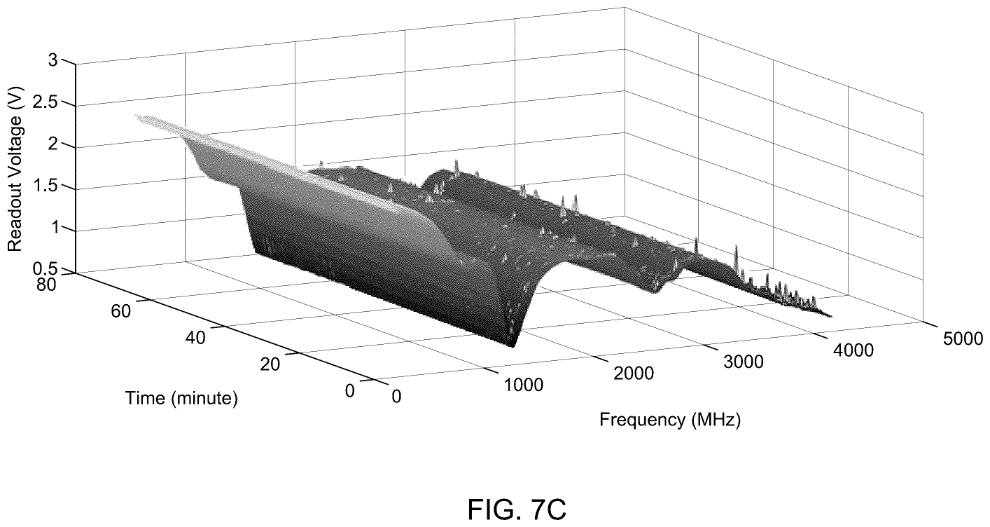

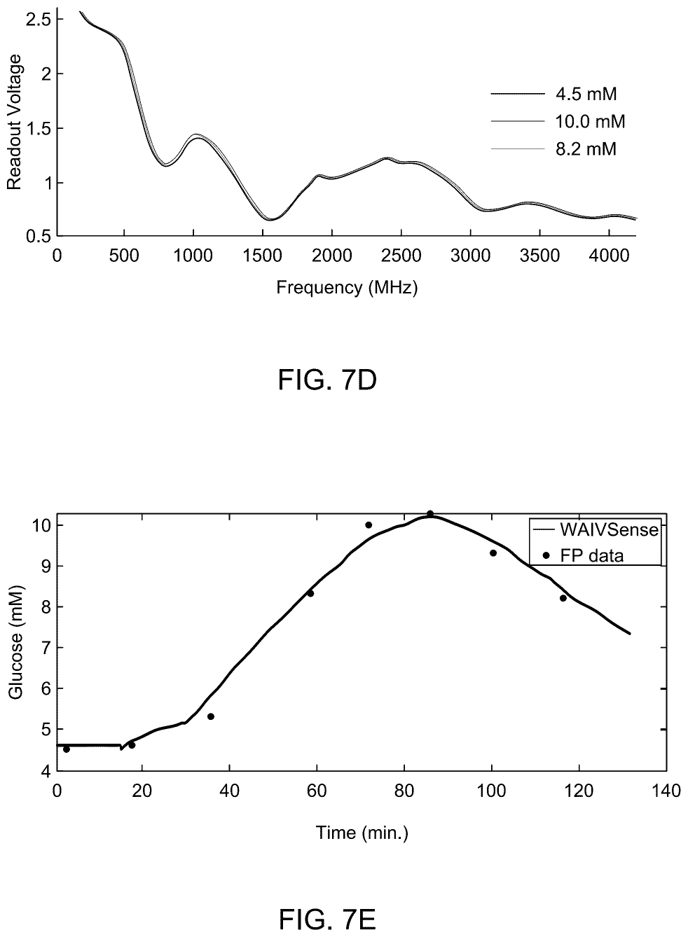

FIG. 7A shows a transmission response of the microwave sensor of over a broad frequency spectrum, and FIG. 7C sets forth a digitized version of the transmission response of FIG. 7A, highlighting variations and interferences as small peaks in the response.

FIG. 7B shows an example plot for results of a human trial lactate measurement using the calibrated non-invasive innovative biosensor, along with a comparison to fingerpick data. The plot (600) demonstrates the well-aligned response of the innovative microwave sensor with the lactate data obtained from fingerpick measurements. This comparison validates the accuracy and reliability of the technology in providing biomarker measurements comparable to traditional invasive methods.

ii. Glucose Monitoring

As another example of the successful implementation of the technology, a human trial was performed in which six (6) healthy individuals volunteered to participate in glucose monitoring. The participants went through a certain procedure approved by the Research Ethics Office at the University of Alberta, Canada.

Participants were expected to attend three laboratory-based sessions. In both sessions, the participants would have their blood glucose level measured every 3-5 minutes using a standard finger-pricking glucometer. At the same time, the participants were wearing the microwave sensor on their wrist using strap bands. Five minutes after the trial initiation, the participants were asked to drink a sugary drink containing 75 grams of glucose to raise their blood glucose level. The participants then rested still until their blood glucose levels dropped back down close to their normal range. The second and third trials for each participant took place on separate days, at least two days after their previous visit, following the same procedure.

The data from the first visit was utilized for building and training/calibrating the AI model. The data from their second visit was used for the verification of the calibration and training of the model. The data from their third visit was used for testing the model and assessing its performance.

FIG. 7D shows an example plot of the microwave data measured by the microwave structure in FIG. 2. FIG. 7E shows an example plot of the results of a human trial glucose measurement using the calibrated non-invasive innovative biosensor, along with a comparison to finger-prick data. The plot demonstrates the well-aligned response of the innovative microwave sensor with the glucose data obtained from finger-prick measurements. This comparison validates the accuracy and reliability of the technology in providing biomarker measurements comparable to traditional invasive methods.

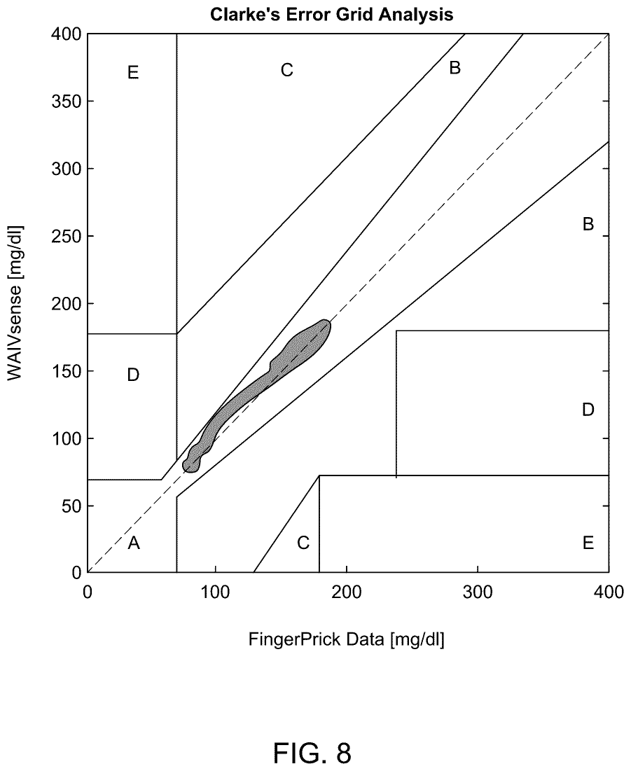

FIG. 8 presents the Clarke Error Grid Analysis for glucose monitoring systems. The presented data are from the human trial for glucose monitoring and are extracted from the results shown in FIG. 8B. As shown, 100% of the calculated data using the calibrated algorithm based on the measurements of the microwave sensing structure are placed in section A, meaning all the measured values are within 20% of the reference standard sensor.

Various systems or methods have been described to provide an example of an embodiment of the claimed subject matter. No embodiment described limits any claimed subject matter and any claimed subject matter may cover methods or systems that differ from those described below. The claimed subject matter is not limited to systems or methods having all of the features of any one system or method described below or to features common to multiple or all of the apparatuses or methods described below. It is possible that a system or method described is not an embodiment that is recited in any claimed subject matter. Any subject matter disclosed in a system or method described that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device. As used herein, two or more components are said to be “coupled”, or “connected” where the parts are joined or operate together either directly or indirectly (i.e., through one or more intermediate components), so long as a link occurs. As used herein and in the claims, two or more parts are said to be “directly coupled”, or “directly connected”, where the parts are joined or operate together without intervening intermediate components.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a pushbutton keyboard, mouse, touchscreen, and the like), and at least one output device (e.g. a display screen, a printer, a wireless radio, and the like) depending on the nature of the device.

It should also be noted that there may be some elements that are used to implement at least part of one of the embodiments described herein that may be implemented via software that is written in a high-level computer programming language such as object-oriented programming or script-based programming. Accordingly, the program code may be written in Java, Swift/Objective-C, C, C++, Javascript, Python, SQL, or any other suitable programming language and may comprise modules or classes, as is known to those skilled in object-oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage. The computer program product may also be distributed in an over-the-air or wireless manner, using a wireless data connection.

The term “software application” or “application” refers to computer-executable instructions, particularly computer-executable instructions stored in a non-transitory medium, such as a non-volatile memory, and executed by a computer processor. The computer processor, when executing the instructions, may receive inputs and transmit outputs to any of a variety of input or output devices to which it is coupled. Software applications may include mobile applications or “apps” for use on mobile devices such as smartphones and tablets or other “smart” devices.

A software application can be, for example, a monolithic software application, built in-house by the organization and possibly running on custom hardware; a set of interconnected modular subsystems running on similar or diverse hardware; a software-as-a-service application operated remotely by a third party; third party software running on outsourced infrastructure, etc. In some cases, a software application also may be less formal, or constructed in ad hoc fashion, such as a programmable spreadsheet document that has been modified to perform computations for the organization's needs.

Software applications may be deployed to and installed on a computing device on which it is to operate. Depending on the nature of the operating system and/or platform of the computing device, an application may be deployed directly to the computing device, and/or the application may be downloaded from an application marketplace. For example, user of the user device may download the application through an app store such as the Apple App Store™ or Google™ Play™.

The present invention has been described here by way of example only, while numerous specific details are set forth herein in order to provide a thorough understanding of the exemplary embodiments described herein. However, it will be understood by those of ordinary skill in the art that these embodiments may, in some cases, be practiced without these specific details. In other instances, well-known methods, procedures and components have not been described in detail so as not to obscure the description of the embodiments. Various modifications and variations may be made to these exemplary embodiments without departing from the spirit and scope of the invention, which is limited only by the appended claims.

1. A non-invasive biometric sensing and monitoring device for determining a characteristic of a state of a user based on a value of a biomolecule within a body tissue, the system comprising:
an encasement member having a set of opposed surfaces offset from one another by a bounding member, a first of the opposed surfaces forming a top surface and a second of the opposed surfaces forming a bottom surface, together the plurality of opposed surfaces and boundary member bounding a cavity, the cavity configured for retaining one or more components of the biometric sensing and monitoring device;
an extended substrate layer positioned within the cavity and being configured for effectuating a coupling between one or more of the retained components:
a frequency synthesizer, coupled to the substrate and being configured for generating a signal to be transmitted;
a microwave structure unit positioned on the extended substrate layer and being coupled to the frequency synthesizer, the microwave structure unit having a first port and a second port, the first port being separated from the second port by an extended segment of a microwave structure of the microwave structure unit, the extended microwave structure segment forming a curvilinear pathway between the first and second ports, each first and second port including a positive and a negative lead separated from one another by a first distance, the first port coupling the frequency synthesizer to the extended microwave structure and being configured for receiving the signal and for transmitting a first portion of the signal linearly forward in a confined manner along the curvilinear pathway of the microwave structure from the first port to the second port, and further being configured for transmitting a second portion of the signal radially forward in a non-confined manner from the first port to the second port so as to produce both a confined signal portion and a non-confined signal portion, the second port being configured for receiving both the confined signal portion and the non-confined signal portion;
a power detector positioned on the extended substrate layer and being coupled to the second port of the microwave structure unit, the power detector being configured for receiving at least a portion of each of the confined and non-confined signal portions, and in response thereto generating both confined and non-confined transmission response data;
an analog to digital converter coupled to the power detector and being configured for converting an analog signal portion of the confined and non-confined transmission response data into digital confined and digital non-confined transmission response data; and
a control unit for directing operations of the frequency synthesized, power detector, and analog to digital converter in accordance with one or more generated or received instructions.

2. The biometric sensing and monitoring device in accordance with claim 1, wherein the extended microwave structure comprises a near field antenna.

3. The biometric sensing and monitoring device in accordance with claim 1, wherein each of the negative leads of the first and second port comprise a solitary ground plane.

4. The biometric sensing and monitoring device in accordance with claim 3, wherein the extended substrate layer comprises a printed circuit board (PCB) being coupled with both excitation circuitry, comprising the frequency synthesizer, and readout circuitry, comprising the power detector, whereby the control unit comprises a processing unit that is configured to control one or more operations of the excitation and readout circuitry via one or more generated control instructions.

5. The biometric sensing and monitoring device in accordance with claim 4, wherein the PCB further comprises a communications module for receiving or communicating the control instructions, whereby the control instructions are configured for initiating generation and transmission of the signal along the microwave structure, and the processing unit is configured for receiving and analyzing, via the readout circuitry, the digital confined and non-confined transmission response data and converting it into confined forward transmission measurement data.

6. (canceled)

7. The biometric sensing and monitoring device in accordance with claim 5, wherein the transmission of the non-confined signal portion forward from the first port to the second port produces a fringe field radiating outward away from the extended microwave structure whereby a portion of the fringe field penetrates into the body tissue at a particularized depth.

8. The biometric sensing and monitoring device in accordance with claim 7, whereby a proportion of the fringe field gets propagated forward and traverses through the body tissue from the first port and is received by the power detector via the second port so as to produce the non-confined signal response data.

9. (canceled)

10. The biometric sensing and monitoring device in accordance with claim 8, whereby one or more of the confined signal response data and non-confined signal response data fluctuate by the presence of one or more biomolecules being present within the body tissue.

11. The biometric sensing and monitoring device in accordance with claim 10, whereby the fluctuation is in response to the concentration of the one or more biomolecules being present within the body tissue.

12. The biometric sensing and monitoring device in accordance with claim 11, whereby the fluctuation is due to a change in dielectric permittivity due to at least one of the one or more biomolecules being present within the body tissue.

13. The biometric sensing and monitoring device in accordance with claim 12, whereby the fluctuation results in a decrease in a measurement of the non-confined signal response data as compared to the confined signal response data.

14. The biometric sensing and monitoring device in accordance with claim 13, wherein the frequency synthesizer is configured for generating a plurality of signals of electromagnetic radiation, each signal of electromagnetic radiation having separate and distinct wavelengths, frequencies, and amplitudes, and further wherein the confined and non-confined signal response data comprises a plurality of confined and non-confined signal response data.

15. The biometric sensing and monitoring device in accordance with claim 14, wherein the communications module is configured for transmitting the plurality of confined and non-confined signal response data to a remote analytics platform via a wireless communications network

16. The biometric sensing and monitoring device in accordance with claim 15, wherein the generation and transmission of the plurality of signals of electromagnetic radiation produces a number of propagated fringe fields each, and further wherein receipt of each of the propagated fringe fields at the power detector produces a broad dielectric spectrum response.

17. The biometric sensing and monitoring device in accordance with claim 16, wherein the remote analytics platform implements a model by which the broad dielectric spectrum response is determined.

18. The biometric sensing and monitoring device in accordance with claim 17, wherein the model employs the broad dielectric spectrum response to generate a distinct dielectric signature for a single biomolecule being present within the body tissue.

19. The biometric sensing and monitoring device in accordance with claim 18, wherein the decrease in the measurement is due to a decrease in received amplitude of the non-confined forward signal response data.

20. The biometric sensing and monitoring device in accordance with claim 19, whereby the model employs the distinct dielectric signature so as to identify the single biomolecule as well as to determine its concentration.

21. The biometric sensing and monitoring device in accordance with claim 20, wherein the distinct dielectric signature comprises a number of complex dielectric characteristics, the complex dielectric characteristics comprising both real and imaginary permittivity.

22. The biometric sensing and monitoring device in accordance with claim 17, wherein the plurality of signals of electromagnetic radiation being transmitted comprises up to 150 signals, each having a different frequency, wavelength, and amplitude.
